(19) United States
(12) Patent Application Publication
(10) Pub. No.: US 2025/0345512 A1
(43) Pub. Date: Nov. 13, 2025

(54) COMBINATION SODIUM-GLUCOSE COTRANSPORTER INHIBITOR (SGLTI) INSULIN THERAPY FOR GLYCEMIC CONTROL IN TYPE 1 DIABETES

(71) Applicant: University of Virginia Patent Foundation, Charlottesville, VA (US)

(72) Inventors: Boris P Kovatchev, Charlottesville, VA (US); Jose Garcia-Tirado, Earlysville, VA (US); Amanda Basu, Charlottesville, VA (US); William Horton, Charlottesville, VA (US)

(21) Appl. No.: 18/847,376

(22) PCT Filed: Mar. 14, 2023
(86) PCT No.: PCT/US2023/015169

§ 371 Date: Sep. 16, 2024

Related U.S. Application Data
(60) Provisional application No. 63/319,667, filed on Mar. 14, 2022.
(60) Provisional application No. 63/320,152, filed on Mar. 15, 2022.

(51) Int. Cl. A61M 5/142; A61K 31/7048

(52) U.S. Cl. CPC A61M 5/142; A61K 31/7048; A61M 2230/201

(57) ABSTRACT

Provided are a method, system, and computer-readable medium for optimizing glycemic control of a diabetic subject having Type 1 diabetes through co-administration of sodium-glucose cotransporter inhibitors (SGLTi) and insulin. Such co-administration can be effected by, for example, regulating one or more administration reactions in view of analyses of continuous glucose monitoring (CGM) data that can be indicative of at least the potential for one or more glycemic events including hypoglycemia and hyperglycemia. The aforementioned regulation can occur according to a balancing of insulin infusion and provisioning of SGLTi so as to avoid the occurrence of either of such events while, at the same time, not promoting an instance of diabetic ketoacidosis (DKA).

### CROSS-REFERENCE TO RELATED APPLICATION

This international application claims priority to and the benefit of U.S. Provisional Application No. 63/319,667, filed Mar. 14, 2022 and U.S. Provisional Application No. 63/320,152, filed Mar. 15, 2022, whereby the entire contents of each of such Applications are incorporated by reference herein.

### STATEMENT OF GOVERNMENT INTEREST

This invention was made with government support under Grant No. DK106785 awarded by The U.S. National Institutes of Health. The government has certain rights in the invention.

### FIELD OF THE DISCLOSURE

Disclosed embodiments relate to mitigating glucose variability by gauging the suitable administration of oral medications that can supplement the provision of insulin by automated delivery regimes, and thus providing for such administration in conjunction with such insulin delivery.

### BACKGROUND

Balancing whether glycemic control can be improved by the combination of conventional automation of insulin delivery and supplementation of oral agents directed to such control has been a topic of study. The discussion below provides pertinent insight.

With the increasing acceptance of continuous glucose monitoring (CGM) for the treatment of type 1 and type 2 diabetes (T1D, T2D), various CGM-based insulin delivery systems became commercially available, including Sensor-augmented pump (SAP) therapies, Low glucose suspend (LGS) systems, Predictive low glucose suspend system (PLGS), and Automated insulin delivery (AID), known as the “artificial pancreas.” The common element of these systems is an insulin pump, which (a) delivers insulin continually based on a pre-programed basal rate and occasional delivery of insulin boluses directed by the patient (i.e., SAP, LGS, PLGS), or (b) automates the insulin delivery process in the case of AID. LGS and PLGS discontinue insulin delivery upon a CGM signaling a low glucose value (e.g., LGS), or predicting low glucose (e.g., PLGS), while AID can lower, discontinue, or increase insulin delivery as needed. Thus, any of these treatment modalities has the potential to discontinue insulin delivery for a certain period of time.

The most sophisticated of these insulin treatment systems—AID—is now commercially available and has improved the management of glycemia in people with T1D. In clinical trials and in real life use, these systems performed better than traditional insulin replacement strategies with respect to obtaining a greater percentage of glucose time-in-range (TIR), lower time in hypoglycemia, and reduced glucose variability.¹⁻⁵ While most AID systems consistently improve overnight control, studies to date, collectively show limitations in achieving optimal daytime control.⁶⁻¹³ This is attributed to primarily meal-related daytime glucose excursions and to the slow action of subcutaneously administered insulin, relative to meal glucose rate of appearance. Even with modern rapid acting insulin analogues, the action of exogenous insulin remains too slow to mitigate postprandial hyperglycemia.¹⁴⁻¹⁶ Several approaches have been tried to improve AID daytime control. New algorithms which attempt to predict meal timing show promise, though they have been only tested in small pilot or in silico studies.¹⁷ Combination therapies use drugs that lower glucose levels or/and slow the appearance of meal carbohydrates in the bloodstream. Agents recently tested with AID include the glucagon-like peptide-1 receptor agonist (GLP-1 RA) liraglutide,¹⁸ exenatide,¹⁹ the dipeptidyl peptidase-4 (DPP-4) inhibitor sitagliptin,²⁰ and the amylin analogue pramlintide.²¹ SGLT2i (i.e., sodium-glucose cotransporter-2 inhibitors) are a newer class of agents that act in an insulin-independent manner to improve glucose control while demonstrating significant cardio-renal benefits in patients with T2D.²² Thus, beyond the expected glycemic benefits of SGLT2i therapy in combination with subcutaneous insulin delivery, it is also anticipated that such treatment may hold invaluable cardiorenal benefits for patients with T1D. There is no current T1D therapy that holds both glycemic and cardiovascular indications from the United States Food and Drug Administration (FDA), so identifying any such treatment would represent a true paradigm shift in clinical practice.

Epidemiological data indicate that people with T1D have lifespans of about 11-13 years shorter than average expectancy²³ and experience cardiovascular disease (CVD) events on average more than a decade earlier than the general population.²⁴ Various studies have identified CVD as the main cause of death in T1D²⁵⁻²⁷ and worrisomely found that T1D confers substantial CVD risk even when conventional treatment targets are achieved.^{28,29} Moreover, diabetic kidney disease is one of the most devastating complications of T1D and is strongly linked to CVD in this population.^{24,30} A recent meta-analysis of SGLT2i therapy in patients with T2D identified significant reductions in both major adverse CVD events and kidney disease progression;³¹ however, such data are currently unavailable in T1D patients. The Empagliflozin as Adjunctive to inSulin thErapy (EASE) trial evaluated 1707 T1D patients and found that 26 weeks of SGLT2i treatment led to significant reductions in body weight, systolic blood pressure, and diastolic blood pressure.³² Sotagliflozin is a dual SGLT1 and SGLT2 inhibitor that the European Medicines Agency has approved as adjunctive therapy to insulin in T1D patients with BMI≥27 kg/m². A recent pooled analysis of 1575 T1D adults treated with sotagliflozin reported short- and long-term renal hemodynamic changes, including reductions in urine albumin:creatinine, that highlight the potential renoprotective effects of this therapy.³³ These positive effects on hypertension, body weight, and renal hemodynamics mirror the well-defined cardiorenal benefits observed with SGLT2i treatment in the T2D population and illustrate the need for further investigation of this therapy in T1D (especially in combination with closed-loop artificial pancreas insulin delivery). One study has examined sotagliflozin in doses of 75, 200, and 400 mg daily in T1D patients, and fount that “sotagliflozin 200 mg and 400 mg improved glycemic control and weight in adults with T1D,” which corresponds to the approval of this medication. Doses of less than 200 mg daily are not currently recommended or used in the clinical practice.³⁴ SGLT2i use as adjuvant therapy to insulin in T1D, however, has been controversial. For example, research on the SGLT2i canagliflozin in T1D has demonstrated improved indices of glycemic variability and improvement in treatment satisfaction versus placebo over 18 weeks.³⁵ However, even with the potential glycemic benefits in T1D, this drug is not approved for the treatment of T1D due to the risk of diabetes ketoacidosis (DKA), where the body produces an insufficient amount of insulin causing a buildup of acids in the bloodstream (i.e., ketones). Dapagliflozin and the dual SGLT1 and SGLT2 inhibitor sotagliflozin, were approved for use in T1D in Europe, but not in the US, due to the lack of sufficient data on increased risk of DKA reported in clinical trials,³² including episodes of euglycemic DKA.^{32,36} Moreover, in 2021, the approval for dapagliflozin for use in type 1 diabetes was withdrawn across Europe and in the UK.

The EASE trials demonstrated improvements in hemoglobin Ale without increase in hypoglycemia, but adverse effects, including DKA, were two to three fold higher with 10 mg and 25 mg empagliflozin daily dose which prompted a recommendation for careful ketone monitoring; in the same studies 2.5 mg empagliflozin had adverse effects indistinguishable from placebo.³² On the other hand, recent data indicate that turning ketogenesis off or on is not affected by SGLT2i use.³⁷ SGLT2i also do not accelerate the rate of ketogenesis following interruption of basal insulin infusion in T1D.³⁸ Overall, there are well documented glycemic benefits of the use of SGLT2i in T1D, but also documented DKA risk associated with the use of these drugs due to reasons that are a matter of debate. Despite elevated risk of DKA, the testing of this class of drugs for the management of T1D in combination with AID is likely to continue, particularly in low doses, given the reported metabolic and cardio-renal benefits as evidenced in the T2D population.²² Recently, two studies added 10 mg bid dapagliflozin³⁹ and 25 mg/day empagliflozin,⁴⁰ to experimental AID systems in two short-term studies (24 h inpatient and 9-14 h outpatient, with study staff on-site, respectively). These studies concluded that this approach may increase time-in-range during full closed-loop admininstration³⁹ and reduce the need for pre-meal carbohydrate counting.⁴⁰

### SUMMARY

It is to be understood that both the following summary and the detailed description are exemplary and explanatory and are intended to provide further explanation of the present embodiments as claimed. Neither the summary nor the description that follows is intended to define or limit the scope of the present embodiments to the particular features mentioned in the summary or in the description. Rather, the scope of the present embodiments is defined by the appended claims.

In view of the above, it would be beneficial to more definitively determine existence and extent of an impact of SGLTi (i.e., sodium-glucose cotransporter-1 and cotransporter-2 inhibitors, respectively), inclusive of one or more of (a) SGLT2 and (b) SGLT1 and SGLT2 (i.e., SGLT1 as accounting for glucose uptake in the intestine, and SGLT2 as accounting for glucose reuptake in the kidney). More particularly, it would be beneficial to examine a formulation of such impact in combination with one or more manner of automated insulin delivery that can optimize one or more reductions in glucose variability.

We have conducted a randomized crossover safety and feasibility study to assess whether daytime glycemic control using a commercially available hybrid AID Control-IQ™ (AID) system or a PLGS Basal-IQ™ (PLGS) system can be improved by a low-dose (5 mg/day) empagliflozin adjuvant therapy.

An embodiment may include a method for controlling a glucose level in a subject having diabetes, the method including providing the subject with an initial dose of sodium-glucose cotransporter inhibitor (SGLTi) lower than a dosing standardized, solely, for treatment of hyperglycemia; analyzing the glycemia level, in real-time, via an automated insulin delivery system comprising continuous glucose monitoring (CGM); providing insulin to the subject via the automated insulin delivery system; and the adjusting insulin delivery, by the automated insulin delivery system, to the subject to maintain the glucose level within a target glucose time-in-range (TIR).

Respective embodiments may further include a relative system and a computer-readable medium commensurate with the embodied method above.

In certain embodiments, the disclosed embodiments may include one or more of the features described herein.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. Embodiments herein will be more particularly described in conjunction with the following drawings wherein:

FIG. 1 illustrates a N-hour decay curve corresponding to insulin-on-board (IOB);

FIG. 2 illustrates, according to embodiments herein, study analysis relative to baseline, control IQ (CIQ), and basal IQ (BIQ) implementation;

FIG. 3 illustrates, according to embodiments herein, area under the curve (AUC) distribution and thresholds for sodium-glucose contransporter inhibitors (SGLTi) adjustment;

FIG. 4A illustrates CIQ relative to time in range (TIR) according to time of day with respect to experimental and control arms (CGM and SGLT2i Empagliflozin, CGM and No SGLT2i Empagliflozin, respectively) according to the study analysis of FIG. 2;

FIG. 4B illustrates CIQ relative to continuous glucose monitoring (CGM) with respect to experimental and control arms according to time of day as to hourly median sensor glucose for the study analysis of FIG. 2;

FIG. 5A illustrates BIQ relative to time in range (TIR) according to time of day with respect to an experimental arm (CGM and SGLT2i Empagliflozin) and a control arm (CGM and No SGLT2i Empagliflozin) according to the study analysis of FIG. 2;

FIG. 5B illustrates BIQ relative to continuous glucose monitoring (CGM) with respect to aforementioned experimental and control arms according to time of day as to hourly median sensor glucose for the study analysis of FIG. 2;

FIG. 6 illustrates the integration of an automated supervisory module (ASM) into one or more existing insulin delivery systems, according to embodiments herein;

FIG. 7 illustrates correlation of an adaptive advisory module (AAM) for the ASM and integrated insulin delivery system of FIG. 6;

FIG. 8 illustrates a high level block diagram of an exemplary ASM-AAM-insulin delivery environment according to embodiments herein;

FIG. 9A illustrates an exemplary computing device which may implement the ASM-AAM-insulin delivery environment;

FIG. 9B illustrates a network system which may implement and/or be used in the implementation of the ASM-AAM-insulin delivery environment;

FIG. 10 illustrates a block diagram which may implement and/or be used in the implementation of the ASM-AAM-insulin delivery environment in association with a connection to the Internet;

FIG. 11 illustrates a system which may implement and/or be used in the implementation of the ASM-AAM-insulin delivery environment in accordance with one or more of a clinical setting and a connection to the Internet; and

FIG. 12 illustrates an exemplary architecture embodying the ASM-AAM-insulin delivery environment.

### DETAILED DESCRIPTION

The present disclosure will now be described in terms of various exemplary embodiments. This specification discloses one or more embodiments that incorporate features of the present embodiments. The embodiment(s) described, and references in the specification to “one embodiment”, “an embodiment”, “an example embodiment”, etc., indicate that the embodiment(s) described may include a particular feature, structure, or characteristic. Such phrases are not necessarily referring to the same embodiment. The skilled artisan will appreciate that a particular feature, structure, or characteristic described in connection with one embodiment is not necessarily limited to that embodiment but typically has relevance and applicability to one or more other embodiments.

In the several figures, like reference numerals may be used for like elements having like functions even in different drawings. The embodiments described, and their detailed construction and elements, are merely provided to assist in a comprehensive understanding of the present embodiments. Thus, it is apparent that the present embodiments can be carried out in a variety of ways, and does not require any of the specific features described herein. Also, well-known functions or constructions are not described in detail since they would obscure the present embodiments with unnecessary detail.

The description is not to be taken in a limiting sense, but is made merely for the purpose of illustrating the general principles of the present embodiments, since the scope of the present embodiments are best defined by the appended claims.

It should also be noted that in some alternative implementations, the blocks in a flowchart, the communications in a sequence-diagram, the states in a state-diagram, etc., may occur out of the orders illustrated in the figures. That is, the illustrated orders of the blocks/communications/states are not intended to be limiting. Rather, the illustrated blocks/communications/states may be reordered into any suitable order, and some of the blocks/communications/states could occur simultaneously.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedure, Section 2111.03.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments. The word “exemplary” is used herein to mean “serving as an example, instance, or illustration.” Any embodiment described herein as “exemplary” is not necessarily to be construed as preferred or advantageous over other embodiments. Additionally, all embodiments described herein should be considered exemplary unless otherwise stated.

It should be appreciated that any of the components or modules referred to with regards to any of the embodiments discussed herein, may be integrally or separately formed with one another. Further, redundant functions or structures of the components or modules may be implemented. Moreover, the various components may be communicated locally and/or remotely with any user/clinician/patient or machine/system/computer/processor. Moreover, the various components may be in communication via wireless and/or hardwire or other desirable and available communication means, systems and hardware. Moreover, various components and modules may be substituted with other modules or components that provide similar functions.

It should be appreciated that the device and related components discussed herein may take on all shapes along the entire continual geometric spectrum of manipulation of x, y and z planes to provide and meet the anatomical, environmental, and structural demands and operational requirements. Moreover, locations and alignments of the various components may vary as desired or required.

It should be appreciated that various sizes, dimensions, contours, rigidity, shapes, flexibility and materials of any of the components or portions of components in the various embodiments discussed throughout may be varied and utilized as desired or required.

It should be appreciated that while some dimensions are provided on the aforementioned figures, the device may constitute various sizes, dimensions, contours, rigidity, shapes, flexibility and materials as it pertains to the components or portions of components of the device, and therefore may be varied and utilized as desired or required.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

Ranges may be expressed herein as from “about” or “approximately” one particular value and/or to “about” or “approximately” another particular value. When such a range is expressed, other exemplary embodiments include from the one particular value and/or to the other particular value.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g., a rat, dog, pig, or monkey), etc. It should be appreciated that the subject may be any applicable human patient, for example.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entireties and to the same extent as if each reference was individually incorporated by reference.

The term “about,” as used herein, means approximately, in the region of, roughly, or around. When the term “about” is used in conjunction with a numerical range, it modifies that range by extending the boundaries above and below the numerical values set forth. In general, the term “about” may be used herein to modify a numerical value above and below the stated value by a variance of 10%. In one aspect, the term “about” may mean plus or minus 10% of the numerical value of the number with which it is being used. Therefore, about 50% may mean in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

As will be understood from the following discussion(s), one or more embodiments herein relate to the treatment of diabetes mellitus and other metabolic disorders, including but not limited to type 1 and type 2 diabetes (T1D, T2D), latent autoimmune diabetes in adults (LADA), postprandial or reactive hyperglycemia, or insulin resistance. In such an embodiment or embodiments, the inventors herein provide for augmenting the action of continuous subcutaneous insulin infusion therapy and related systems, such as sensor-augmented pump (SAP), low glucose suspend (LGS), predictive low glucose suspend (PLGS), or automated insulin delivery (AID), known as the “artificial pancreas,” by providing additional mitigation of glucose variability according to administration of oral medications, such as sodium-glucose cotransporter inhibitors (SGLTi), including one or more of (a) a SGLT2 inhibitor and (b) a combination SGLT1 and SGLT2 inhibitor. For example, one or more of discussed embodiments demonstrates that daytime glycemic control using a commercially available hybrid AID Control-IQ™ (AID) system or a PLGS Basal-IQ™ (PLGS) system can be improved by a low-dose (5 mg/day) empagliflozin adjuvant therapy.

In these regards, one or more embodiments herein address providing for relevant interaction between the following, including: (1) a sub-recommended initial dose of SGLTi added to insulin delivery as an oral supplement, whereafter this dose may be adjusted by an adaptive advisory module (AAM) discussed herein, though such adjusted dose is contemplated to remain within limits lower than those prescribed solely according to clinical practice for the control of diabetes due to administration of SGLTi alone; (2) an automated supervisory module (ASM) that works as a superstructure to any insulin delivery system based on continuous glucose monitoring (CGM), so as to prevent such system from depriving a person from active insulin for a certain period of time (e.g., 30 minutes) in order to thereby reduce the risk of euglycemic DKA that can be typically associated with the stand-alone use of SGLTi for the treatment of diabetes; and (3) the aforementioned AAM that is operative to observe the magnitude of postprandial glucose excursions via CGM in order to suggest SGLTi dose changes as needed (relative to one or more of maintenance of TIR and decrease of glucose variability). Herein, it is contemplated that an insulin delivery system may describe (a) sensor-augmented pump (SAP) therapy; (b) a low glucose suspend (LGS) system or predictive low glucose suspend system (PLGS); or (c) an automated insulin delivery (AID), known as the “artificial pancreas.” When implemented as AID, the ASM may direct a patient to adjust her insulin pump basal rate and automatically adjust control algorithm parameters, as appropriate, so as to at least maintain TIR. When in conjunction with SAP, LGS, or PLGS, the ASM may be operative to suggest to a patient adjustments to her insulin pump basal rate settings. Herein, the term “SGLTi” can mean a SGLT2i medication or a combination medication including one or more of SGLT1 and SGLT2 inhibitors.

Embodiments herein providing for the above-referenced interactions are directed to resolving, as shown through herein described study, a dichotomy presented by use of insulin alone relative to SGLTi use alone. That is, it is understood that insulin delivery systems are typically most effective in a steady state, e.g., when a person sleeps at night, and are least effective in post-meal state when subcutaneously injected insulin is typically too slow to mitigate postprandial glucose excursions. In contrast, SGLTi medications are designed to target and attenuate postprandial hyperglycemia. In other words, such embodiments, as discussed with referenced to the aforementioned study, can demonstrate that co-administration of SGLTi and insulin can effectuate at least one or more of increased TIR and decreased glucose variability.

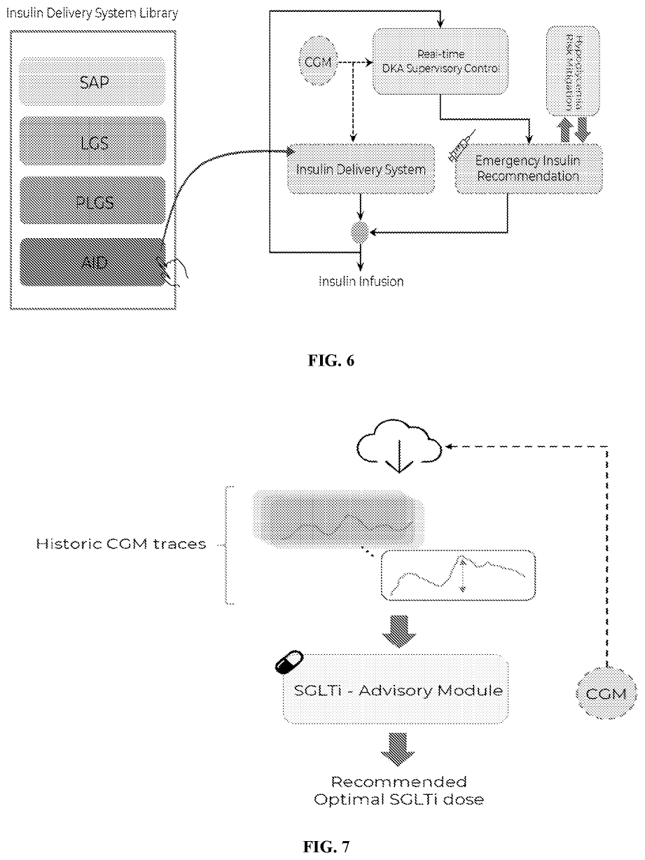

Referring to FIG. 6, functionality for the integration of ASM regarding a respective insulin delivery system, according to embodiments herein, is provided. In this regard, an insulin delivery system library 610 comprising SAP, LGS, PLGS, and AID is provided, in which, according to a user provision, AID is discussed herein for exemplary explanation. That is, such AID may effectuate an insulin delivery system according to CGM, whereas the ASM 620 is integrated therewith to monitor CGM readings in real-time to prevent DKA of a relevant patient. To achieve such prevention, one or more embodiments herein contemplate the ASM 620 pushing an adjustment amount to the AID as an emergency insulin recommendation (EIR) according to the corresponding EIR module 630.

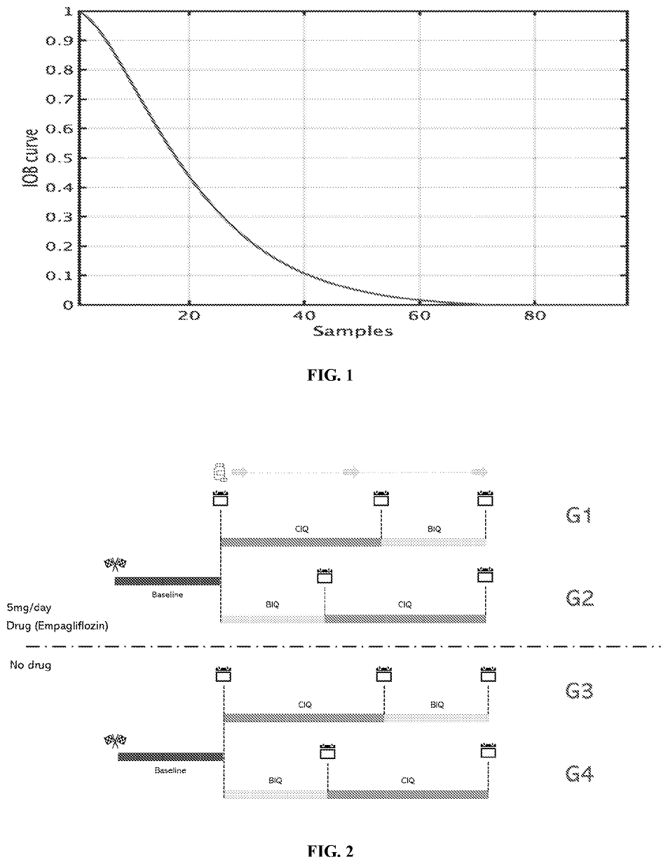

Here, such recommendation can be a function of the ASM's determination and evaluation of insulin-on-board (IOB), which can be understood as a mathematical construct allowing estimation of the amount of circulating insulin that is still active in the circulation and which has not been cleared by a subject. IOB can often be described mathematically by a N-hour decay curve (see, e.g., FIG. 1), where N is regarded as a design parameter. As an example, IOB can be described below, with N=6,

$$IOB = ( \begin{matrix}{ic}_{0} & {ic}_{1} & {ic}_{2} & … & {ic}_{71}\end{matrix} ) ( \begin{matrix}δ {u}_{0} \\ δ {u}_{- 1} \\ δ {u}_{- 2} \\ ⋮ \\ δ {u}_{- 71}\end{matrix} ) ,$$

where ic₀, . . . , ic₇₁ represents the sequence describing the decaying curve and δu₀, . . . , δu₋₇₁ represents a vector of past insulin injections in 5-min increments. The ASM 620 can, in real-time, function as a dynamic constraint generator according to a function of the current IOB, last 1-h CGM, and insulin sequence,

$${\{ CGM \}}_{k - T}^{k} and {\{ {u}_{i} \}}_{k - T}^{k} ,$$

respectively, according to the following:

$$ASM = f ( IOB , {\{ CGM \}}_{k - T}^{k} , {\{ {u}_{i} \}}_{k - T}^{k} ) .$$

As a result, ASM 620 can determine an appropriate basal rate to be administered and provide the same to EIR module 630. Upon receipt, EIR module 630 may conduct a hypoglycemia risk mitigation analysis 640. For example, the analysis may ensure that the basal rate does not cause glucose to fall below 70 mg/dL. A result of the analysis can be a finally determined basal rate targeted at avoiding DKA and hypoglycemia.

In one or more embodiments herein, the aforementioned co-administration of SGLTi and insulin can include providing to a subject an initial oral daily dose of SGLTi in amounts of about 25 to about 50 percent of a minimal dosage ordinarily used, i.e., standardized, in clinical practice relative to treatment for glucose variability such as postprandial excursion. In these regards, such dosing may be relative to a particular SGLTi being contemplated. Herein, we have analyzed the following dosing that may be particularly beneficial, including: (a) SGLT2i empagliflozin between about 2.5 to about 5 mg/daily, where previously higher doses of 10 to 25 mg/daily were used as clinically recommended, i.e., standardized; (b) SGLT2i dapagliflozin between about 2.5 to about 5 mg daily, where previously higher doses of 10 mg/daily were used as clinically recommended; and (c) dual SGLT1 and SGLT2 inhibitor sotagliflozin between about 100-150 mg/daily, where previously higher doses of 200-400 mg/daily were used as clinically recommended.

In other words, through implementation of ASM 620 and EIR module 630 operating to govern insulin infusion in view of initial SLGTi dosing, we have demonstrated that the herein discussed co-administration of SGLTi and insulin provides, as now described, at least a two-fold advantage over known, singular insulin therapies. First, such dual administration results in improved action of subcutaneously injected insulin in the postprandial state due to SGLTi action, thereby achieving several therapeutic advantages including the following: (i) reduction of postprandial glucose excursions and glucose variability to a degree higher that the degree achievable by insulin alone; (ii) reduction in glycosylated hemoglobin (HbA1c—a universally accepted metric of glycemic control in diabetes), to a degree higher that the degree achievable by insulin alone; (iii) improvement in CGM-measured time-in-range (TIR, typically the percent time a patient spends within the target range 70-180 mg/dL), to a degree higher that the degree achievable by insulin alone; (iv) compounding of the cardiovascular benefits of reduced glucose variability and the cardiovascular benefits of SGLTi, thereby achieving a lower overall risk of cardiovascular complications frequently observed due to diabetes. Second, such co-administration has revealed reduced negative side effects of SGLTi, due to the use of lower doses, as evinced most prominently in reduction of the risk for DKA, which is the most significant deterrent of use of SGLT2i in T1D.

In order to optimize effectiveness of SGLTi dosing, while the effect thereof on insulin supply can still be managed according to ASM 620 and EIR module 630, one or more embodiments herein further incorporate the hereinabove discussed adaptive advisory module (AAM). In this regard, and when referring to FIG. 7, ongoing dosing recommendation can be formulated to address postprandial glucose excursion, thus enabling maintaining an intended effect of SGLTi co-administration. In other words, one or more embodiments herein contemplate continual identification and monitoring of an extent of postprandial excursion, and depending on degree, suggesting at least one modification to an initial dosing of SGLTi.

As shown in FIG. 7, the AAM 720 (SGLTi-Advisory Module therein) can implement a real-time cloud (i.e., internet) connection among or to AIM of FIG. 6 to examine CGM data of a subject, and particularly last Nₜᵣ daily CGM and insulin profiles, with Nₜᵣ∈ [symbol] being a design parameter. The previous profiles can be analyzed in terms of peak postprandial CGM 710 (postprandial excursion) for all detected meals to assess adequacy of the currently administered SGLTi dosing. Metrics that can used to quantify the magnitude of postprandial excursions can be at least one of the following: Area under the Curve (AUC), Standard Deviation of CGM glucose (SD) and Coefficient of Variation of CGM glucose (CV), Rate of Change of CGM glucose, High Blood Glucose Index (HBGI), Hourly Risk Range (HRR), Mean Amplitude of Glucose Excursions (MAGE), Mean Absolute Glucose Change (MAG), Continuous Overlapping Net Glycemic Action (CONGA), or other metrics of hyperglycemic excursions used for analysis of diabetes control, according to studied review of the same.⁴¹ One embodiment using AUC as a metric of postprandial excursions is presented below.

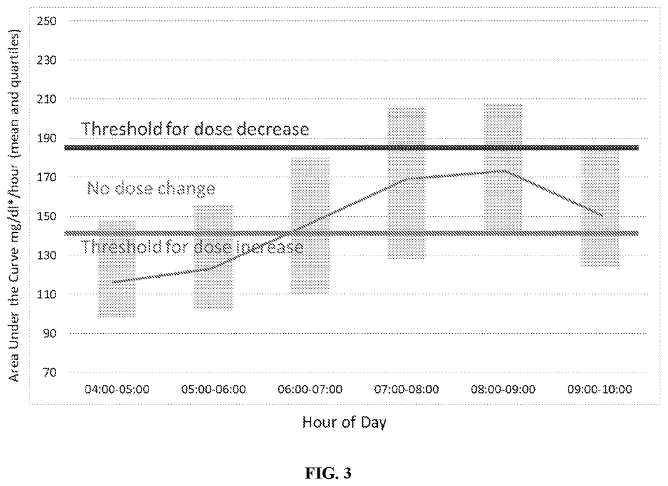

In alternative embodiments using other metrics, the formulas will remain similar, but will replace AUD with alternative metrics and action thresholds (such thresholds being exemplified according to FIG. 3, for example). Mathematically, the AAM 720 can detect meal excursions (e.g., nadir of meal-related CGM excursions) for the last Nₜᵣ CGM traces; compute the AUC corresponding to all meal-related CGM excursions; assess the distribution of all computed AUCs in terms of mean, standard deviation, and quartiles; and compute the SGLTi dose according to

$${SGLTi}_{dose} = \{ \begin{matrix}current dose & if μ ( AUC ) \leq {AUC}_{th} \\ scale - up & if AUC > {AUC}_{th} \\ scale - down & if K \geq {K}_{th} or {N}_{h} > 2\end{matrix} ,$$

where μ(AUC) represents the averaged area-under-the curve over the last Nₜᵣ days according to CGM trace, AUCₜₕ represents a design AUC threshold,

$$K = g ( IOB , {\{ CGM \}}_{k - T}^{k} , {\{ {u}_{i} \}}_{k - T}^{k} )$$

represents a daily ketone estimation with Kₜₕ=1.5 mmol/L, and Nₕ is the number of hypoglycemic episodes over the last Nₜᵣ days that could be computed as a surrogate (i.e., a value) that can correspond to the time spent below 70 mg/dL.

FIG. 2 presents a study analysis relative to baseline, control IQ (CIQ) (e.g., AID), and basal IQ (BIQ) (e.g., PLGS) implementation, according to investigation that is demonstrable of one or more embodiments herein. Therein, an experimental arm (G1, G2) providing an initial daily dose of 5 mg empagliflozin (EMPA) is depicted against a control arm (G3, G4) without such dosing (NOEMPA). Relative characteristics of the analysis are provided below.

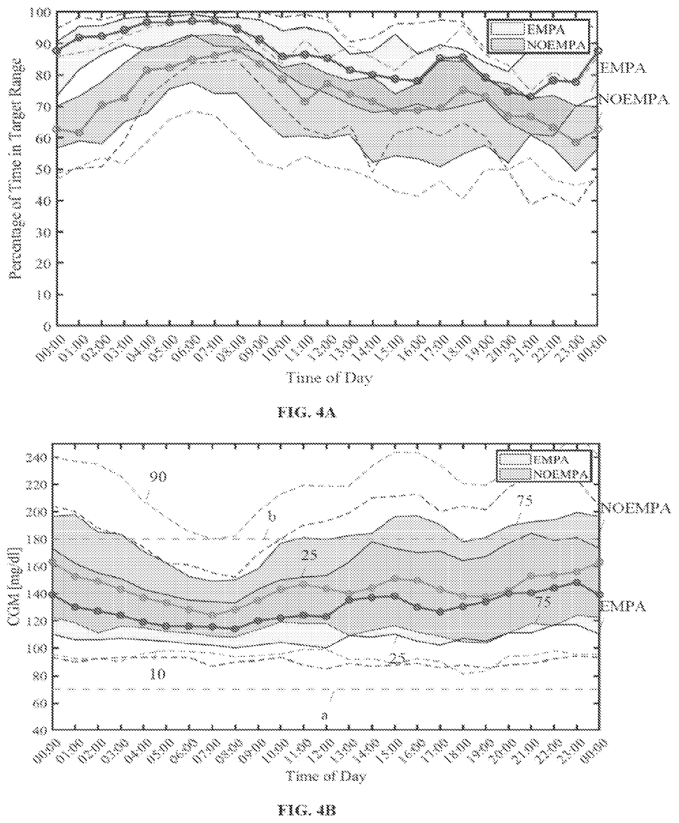

Study Design: A single-center, randomized, controlled, unblinded, crossover clinical trial (NCT04201496) compared four parallel groups, two groups in the experimental arm (EMPA) with 5 mg daily empagliflozin as adjunctive therapy to PLGS or AID, and two groups in the control arm (NOEMPA) with no drug and with a PLGS system (Basal-IQ, Tandem Diabetes Care) or AID system (Control-IQ, Tandem Diabetes Care). The groups within each arm differed from one another in the order of the randomly assigned crossover technological intervention, relative to PLGS being followed by AID or AID being followed by PLGS, respectively (FIG. 4). The study protocol was approved by the University of Virginia Institutional Review Board (IRB). An investigational device exemption (IDE), including an investigational new drug (IND) application for empagliflozin, was approved by the FDA. Safety aspects were overseen by an external data and safety monitoring board (DSMB).
Participants: Major inclusion criteria were T1D treated with insulin for at least one year, use of continuous subcutaneous insulin infusion therapy for at least six months, age 18-65 yrs., no use of glucose lowering agents other than insulin, and Hb1Ac<9% (75 mmol/mol).
Randomization: A (1:1:1:1) randomization assigned participants to either Group 1, AID-EMPA (4 weeks) followed by PLGS-EMPA (2 weeks), Group 2, PLGS-EMPA (2 weeks) followed by AID-EMPA (4 weeks), Group 3, AID-NOEMPA (4 weeks) followed by PLGS-NOEMPA (2 weeks), and Group 4, PLGS-NOEMPA (2 weeks) followed by AID-NOEMPA (4 weeks).
Procedures: After confirmation of eligibility, participants underwent study equipment and medication training (experimental arm), or only study equipment training (control arm). Study equipment consisted of Dexcom G6® CGM (Dexcom Inc., San Diego, CA), Contour® Next blood glucose (Ascensia Diabetes Care, Basel, Switzerland) and Precision Xtra® blood ketone meters (Abbott, Alameda, CA) with their respective test strips; infusion sets, and the use of the t:slim X2™ insulin pump with Basal-IQ or Control-IQ technologies (Tandem Diabetes Care, San Diego, CA), according to randomization. The trial had a run-in phase to collect baseline CGM data and to train participants on the use of study devices. Participants in the experimental arm were asked to record two days of baseline ketone values before initiating the use of the study drug. Thereafter, participants were directed to use the study CGM with EMPA for 1-2 weeks. A tolerance to the medication was assessed prior to the starting of the next study phase, including adherence to the protocol, ketone testing 2-4 times per day with monitored values not greater than 0.6 mmol/L on at least two successive occasions (the first testing upon waking in a fasted state), no adverse events relating to perineal infection or symptomatic postural hypotension, and no evidence of significant hypoglycemia <54 mg/dL, or any other listed adverse effects of the medication. Participants in the control arm were directed to use the study CGM for 1-2 weeks.
Sample Size: The total sample size was projected to be 40 participants, assuming 1) 1:1:1:1 randomization; 2) 90% power and type 1 error a=0.01, further reinforcing the feasibility of hierarchical analyses. The projected recruitment sample was n=50, to accommodate up to 20% attrition rate without sacrificing statistical power.
Analytical methods: For the primary analysis, TIR during the day in the 4-week AID period was compared between the two EMPA groups versus the two NOEMPA groups using a linear mixed-effects regression model while adjusting for pre-randomization HbAlc and age. Analyses of the secondary outcomes were conducted by the same method that was used in the primary analysis.
Results: Between August of 2020 and August of 2021, 39 volunteers signed the study consent form. The entire trial was completed by 32 (89%) participants. Demographic characteristics are shown in Table 1 below. At the end, 34 participants were considered for the primary end-point and safety assessments. Study participants performed a median of one and three daily fingerstick ketone measurements in the non-drug groups and drug groups.

TABLE 1
Demographics

| | EMPA | NOEMPA | Total |
|---|---|---|---|
| | n = 18 | n = 17 | n = 35 |
| Age (years) | 40 ± 14 | 42 ± 13 | 41 ± 14 |
| Diabetes duration (years) | 21 ± 13 | 21 ± 13 | 21 ± 13 |
| BMI (kg/m²) | 30 ± 6 | 29 ± 5 | 29 ± 5 |
| Creatinine (mg/dL) | 0.82 ± 0.17 | 0.84 ± 0.21 | 0.83 ± 0.19 |
| eGFR (mL/min/1.73 m²) | 91.5 ± 24.5 | 86.5 ± 25.5 | 89.1 ± 24.7 |
| Gender (F:M) | 13:5 | 11:6 | 24:11 |
| Previous pump use (years) | 13.1 ± 7.9 | 12.7 ± 7.3 | 13.2 ± 7.8 |

Race/Ethnicity

| | | | | | | |
|---|---|---|---|---|---|---|
| White - no/total no. (%) | 17/18 | (94.4) | 16/17 | (94.1) | 33/35 | (94.3) |
| Hispanic or Latino ethnic group - no. (%) | 1 | (5.6) | 0 | (0) | 1 | (2.9) |
| African American - no. (%) | 0 | (0) | 1 | (5.9) | 1 | (2.9) |

HbA1c

| | | | |
|---|---|---|---|
| | 6.7 ± 1 | 7.1 ± 1 | 6.8 ± 0.9 |
| mmol/mol | 50 ± 11 | 54 ± 11 | 52 ± 10 |

Abbreviations: BMI, body mass index; EMPA, Empagliflozin.

Primary End Point: Mean±SD percent time in target range 70-180 mg/dL (3.9-10 mmol/L) TIR during daytime (7:00 to 23:00 h) was 81+10 in the AID-EMPA arm vs 71+10 in the AID-NOEMPA arm, with a mean adjusted difference of +9.9 percentage points [95% CI 0.6 to 19.1] (amounting to 1.6 hours per day); p=0.04, as shown in Table 2 below and FIG. 4A, where TIR envelopes (median and interquartile range) are shown for each hour of the day.

TABLE 2
Primary and Secondary Hierarchical Efficacy Outcomes*

| | | | CIQ-EMPA vs. | |
|---|---|---|---|---|
| | CIQ- | CIQ- | CIQ-NOEMPA | |
| | EMPA | NOEMPA | Difference | p- |
| Outcome | n = 17 | n = 16 | (95% CI)§ | value |
| Primary: daytime glucose % Time in | 81 ± 10 | 71 ± 10 | 9.9(0.6 to 19.1) | 0.04 |
| range of 70 to 180 mg/dL (3.9-10.0 | | | | |
| mmol/L) | | | | |
| Secondary hierarchical outcomes | | | | |
| in prespecified order ¶ | | | | |
| 24/7 glucose level <70 mg/dL (<3.9 | 1.1(0.5-1.5) | 1.9(0.7-3.7) | −1.1(−3.2 to 0.9) | 0.21 |
| mmol/L) - median % time (IQR)** | | | | |
| 24/7 Mean glucose level - mg/dL | 137 ± 19 | 154 ± 18 | −17.3(−35.4 to 0.8) | NA |
| Daytime CGM SD - mg/dL | 42.6 ± 9.8 | 52 ± 9.9 | −9.3(−17.8 to −0.93) | NA |
| Daytime CGM CV - % | 30.4 ± 3.5 | 34 ± 3.8 | −3.3(−6.1 to −0.5) | NA |
| 24/7 LBGI - median (IQR)** | 0.5(0.4-0.6) | 0.5(0.3-1) | 0.06(−0.4 to 0.5) | NA |
| 24/7 HBGI - median (IQR)** | 2.7(2-4.4) | 5.6 | −2.5(−5.3 to 0.2) | NA |

*Plus-minus values are means ± SD. All the subjects were included in the model on an intention-to-treat basis. Missing data were handled by means of direct likelihood analyses. To convert the values for glucose to millimoles per liter, multiply by 0.05551. NA denotes not applicable.
Baseline outcomes for 18 of the 35 subjects were not available.
The baseline glycated hemoglobin level was measured at the randomization visit.
§Differences were calculated as percentage points (the value in the treatment -EMPA- minus the value in the control -NOEMPA- group) and were model-adjusted for the pre-randomization value of the HbA1c.
¶ To control the type 1 error, a hierarchical approach was used in which hypothesis testing was performed sequentially in the order listed in the table. When a p value of 0.05 or higher was observed, the outcomes below that finding on the list were not formally tested.
**Distributions were skewed and were thus modeled with the use of rank-based transformation.

Secondary End Points: Median interquartile range (IQR) percent time that the glucose level was <70 mg/dL (3.9 mmol/L) 24/7 was 1.1(0.5-1.5) in the AID-EMPA arm versus 1.9(0.7-3.7) in the AID-NOEMPA arm, with a mean adjusted difference of −1.1 percentage points [95% CI −3.2 to 0.9]; p=0.21. The 24/7 mean glucose level in both the experimental and control groups resulted as 137 mg/dL versus 154 mg/dL (Table 2 above).

AID-EMPA outperformed PLGS-EMPA in terms of percentage of TIR overnight, with a mean adjusted difference of +10.2 percent points [95% confidence interval (CI) 3.6 to 16.8];

p=0.004, meaning that AID-EMPA kept the study subjects 7 h out of 8 h of the night-time in range vs 6 h out of 8 h for PLGS-EMPA. Additionally, AID-EMPA was superior to PLGS-EMPA in terms the percentage time <70 mg/dL, with a mean adjusted difference of −1.5 percent points [95% CI −2.6 to −0.3]; p=0.01, and percentage time >180 mg/dL, with a mean adjusted difference of −9.5 percent points [95% CI −16 to −2.9]; p=0.006. During the daytime and overall, AID-EMPA was superior to PLGS-EMPA only in the percentage time <70 mg/dL, with mean adjusted differences of −1 and −1.2 percentage points [95% CI −2 to 0]; p=0.05 and [95% CI −1.8 to −0.6]; p<0.001, respectively. In addition, PLGS-EMPA reached a decrease of 24 mg/dL in mean glucose overall and 18.6% decrease in percent time in hyperglycemia (see Tables 4 and 5 below). Overnight, PLGS-EMPA, compared to PLGS-NOEMPA, showed 17% increase in percent TIR and 7.6% decrease in time in hyperglycemia (see Table 6 below). Similarly to AID, the infused insulin amounts were lower in PLGS-EMPA than in PLGS-NOEMPA 24/7 and overnight, which corresponded to an insulin decrease of 25% and 40%, respectively.

TABLE 4
Glycemic outcomes overall.

| | CIQ-EMPA | | | CIQ-NOEMPA | | | BIQ-EMPA | | | BIQ-NOEMPA | | |
|---|---|---|---|---|---|---|---|---|---|---|---|---|
| | n = 16 | | | n = 16 | | | n = 16 | | | n = 16 | | |
| Glycemic metrics | | | | | | | | | | | | |
| Mean CGM | 137 ± 18.6 | | | 1.54 ± 18 | | | 1.41 ± 30 | | | 165 ± 31.8 | | |
| glucose (mg/dL) | | | | | | | | | | | | |
| % CGM time | 0(0-0.2) | | | 0.3(0.1-0.5) | | | 0(0-0.4) | | | 0.14(0-0.5) | | |
| <50 mg/dL | | | | | | | | | | | | |
| (<2.8 mmol/L)** | | | | | | | | | | | | |
| % CGM time | 0.3(0.1-0.5) | | | 0.8(0.3-1.4) | | | 0.5(0.2-0.9) | | | 0.5(0.1-1.7) | | |
| <60 mg/dL | | | | | | | | | | | | |
| (<3.3 mmol/L)** | | | | | | | | | | | | |
| % CGM time | 1.1(0.5-1.5) | | | 1.9(0.7-3.7) | | | 1.7(0.9-2.7) | | | 1.1(0.6-5.1) | | |
| <70 mg/dL | | | | | | | | | | | | |
| (<3.9 mmol/L)** | | | | | | | | | | | | |
| % CGM time | 59.6 ± 13.7 | | | 4.5 ± 11.7 | | | 56.8 ± 16.5 | | | 40 ± 16.2 | | |
| <70-140 mg/dL | | | | | | | | | | | | |
| (<3.9-7.8 mmol/L) | | | | | | | | | | | | |
| % CGM time | 83 ± 10.6 | | | 71 ± 9.7 | | | 79 ± 15.7 | | | 62.2 ± 16 | | |
| <70-180 mg/dL | | | | | | | | | | | | |
| (<3.9-10.0 mmol/L) | | | | | | | | | | | | |
| % CGM time | 11.5(7-19) | | | 25.6(19.3-34.3) | | | 15(6.7-22.7) | | | 32(18.3-46.6) | | |
| >180 mg/dL | | | | | | | | | | | | |
| (>10.0 mmol/L)** | | | | | | | | | | | | |
| % CGM time | 1.1(0.2-2.9) | | | 4.4(2.9-7.1) | | | 1.7(0.2-3.6) | | | 8.9(2.9-14) | | |
| >250 mg/dL | | | | | | | | | | | | |
| (>13.9 mmol/L)** | | | | | | | | | | | | |
| % CGM time | 0(0-0.5) | | | 1(0.3-2.6) | | | 0.2(0-0.6) | | | 2.7(0.2-6) | | |
| >300 mg/dL | | | | | | | | | | | | |
| (>16.7 mmol/L)** | | | | | | | | | | | | |
| CGM SD (mg/dL) | 40.5 ± 9.4 | | | 52.6 ± 9.8 | | | 46 ± 14.6 | | | 60.2 ± 12.1 | | |
| CGM CV (%) | 29.4 ± 3.6 | | | 34 ± 3.7 | | | 32.1 ± 3.7 | | | 36.6 ± 3.1 | | |
| CGM LBGI** | 0.5(0.4-0.6) | | | 0.5(0.3-1) | | | 0.7(0.6-1) | | | 0.5(0.3-1.3) | | |
| CGM HBGI** | 2.7(2-4.4) | | | 5.6 | | | 3.3(1.8-4.7) | | | 7.6(3.8-10.6) | | |
| Safety Metrics | | | | | | | | | | | | |
| Ketones | 0.20 ± 0.05 | | | 0.14 ± 0.08 | | | 0.20 ± 0.05 | | | 0.14 ± 0.06 | | |
| (mmol/L)** | | | | | | | | | | | | |
| Technical | | | | | | | | | | | | |
| performance | | | | | | | | | | | | |
| metrics | | | | | | | | | | | | |
| Injected Insulin | 0.52 ± 0.15 | | | 0.71 ± 0.26 | | | 0.50 ± 0.12 | | | 0.65 ± 0.26 | | |
| (IU/kg · day) | | | | | | | | | | | | |
| | CIQ-EMPA vs. | | BIQ-EMPA vs. | | CIQ-EMPA vs. | | CIQ-EMPA vs. | | BIQ-EMPA vs. | | CIQ-NOEMPA vs. | |
| | CIQ-NOEMPA | | BIQ-NOEMPA | | BIQ-NOEMPA | | BIQ-NOEMPA | | CIQ-NOEMPA | | BIQ-NOEMPA | |
| | Difference | p- | Difference | p- | Difference | p- | Difference | p- | Difference | p- | Difference | p- |
| | [symbol] | value | [symbol] | value | [symbol] | value | [symbol] | value | [symbol] | value | [symbol] | value |
| Glycemic metrics | | | | | | | | | | | | |
| Mean CGM | [symbol] | 0.051 | [symbol] | 0.01 | [symbol] | 0.371 | [symbol] | 0.003 | [symbol] | 0.14 | [symbol] | 0.02 |
| glucose (mg/dL) | | | | | | | | | | | | |
| % CGM time | −0.2(−0.4 | 0.23 | 0(−0.3 | 0.82 | 0.1(−0.1 | 0.22 | 0.1(−0.2 | 0.38 | 0.3(−0.5 | 0.05 | 0.2(0 | 0.02 |
| <50 mg/dL | to 0.1) | | to 0.2) | | to 0.3) | | to 0.4) | | to 0) | | to 0.4 | |
| (<2.8 mmol/L) | | | | | | | | | | | | |
| % CGM time | [symbol] | 0.13 | 0(−1 | 0.93 | −0.5(−0.9 | 0.033 | −0.5(−1.5 | 0.25 | 0.2(−1.2 | 0.62 | 0.2(−0.3 | 0.39 |
| <60 mg/dL | | | to 0.9) | | to 0) | | to 0.4) | | to 0.3) | | to 0.6) | |
| (<3.3 mmol/L) | | | | | | | | | | | | |
| % CGM time | −2.1(−3.2 | 0.26 | 0.2(−1.7 | 0.84 | −1.2(−2 | 0.0087 | −1(−2.9 | 0.35 | 0.1(−1.8 | 0.99 | 0.1(−6.4 | 0.63 |
| <70 mg/dL | to 0.9) | | to 2.1) | | to −0.3) | | to 0.9) | | to 2) | | to 0.7) | |
| (<3.9 mmol/L) | | | | | | | | | | | | |
| % CGM time | 14.7(4.1 | 0.008 | 16.0(6.3 | 0.002 | 2.8(−1.8 | 0.22 | 19.7(9.2 | <0.001 | 11.8(1.3 | 0.029 | [symbol] | 0.034 |
| 70-140 mg/dL | to 25.2) | | to 27.4) | | to 7.5) | | to 30.2) | | to 22.4) | | | |
| (<3.9-7.8 mmol/L) | | | | | | | | | | | | |
| % CGM time | 12(2.5 | 0.015 | 16.7(7.2 | 0.001 | 4.2(−0.7 | 0.089 | 21(11.3 | <0.001 | 7.8(−1.7 | 0.081 | 8.9(4 | <0.001 |
| 70-180 mg/dL | to 21.5) | | to 26.3) | | to 9) | | to 10.4) | | to 12.4) | | to 13.7) | |
| (<3.9-10.0 mmol/L) | | | | | | | | | | | | |
| % CGM time | −22.3 | 0.018 | −18(−28.7 | <0.001 | −3.4(−8.3 | 0.17 | −22(−32.1 | <0.001 | −8.9(−19 | 0.002 | 9.7(−14.7 | <0.001 |
| >180 mg/dL | (22.4 | | to −8.5) | | to 1.6) | | to 11.9) | | to 1.2) | | to −4.7) | |
| (>10.0 mmol/L) | to −2.2) | | | | | | | | | | | |
| % CGM time | −4.9(−8.1 | 0.004 | −5.5(−8.7 | 0.001 | 0.2(−0.9 | 0.76 | −5.4(−8.5 | 0.002 | −3(−8.3 | 0.093 | −0.5(−1.6 | 0.37 |
| >250 mg/dL | to −1.6) | | to −2.3) | | to 1.2) | | to −2.1) | | to −1.8) | | to 0.6) | |
| (>13.9 mmol/L) | | | | | | | | | | | | |
| % CGM time | −2.2(−4.9 | 0.095 | −3.4(−6.1 | 0.012 | −1.6(−3.7 | 0.11 | −5.1(−3.7 | <0.001 | −0.6(−3.2 | 0.65 | −2.8(−1.9 | 0.009 |
| >300 mg/dL | to 0.4) | | to −0.8) | | to 0.4) | | to −2.4) | | to 2) | | to −0.7) | |
| (>16.7 mmol/L) | | | | | | | | | | | | |
| CGM SD (mg/dL) | −12.3 | 0.005 | −14.3 | 0.001 | −5.5(10 | 0.020 | −19.7(−28 | <0.001 | −6.7(−15 | 0.11 | −7.6(−12.1 | 0.002 |
| | (−29.5 | | (−22.6 | | to 0.9) | | to 11.4) | | to 1.6) | | to −3) | |
| | to 3.8) | | to 5.9) | | | | | | | | | |
| CGM CV (%) | −4.7(−7.2 | <0.001 | −4.5(−7 | <0.001 | −2.7(−4.5 | 0.004 | −7.2(−9.8 | <0.001 | −1.9(−4.5 | 0.10 | −2.6(−4.3 | 0.006 |
| | to 2.1) | | to −2) | | to 1) | | to −4.7) | | to 0.6) | | to −0.8) | |
| CGM LBGI | 0(−6.6 | 0.71 | 0.1(0.3 | 0.46 | −0.3(−0.3 | 0.001 | −0.1(−0.6 | 0.74 | 0.2(0.2 | 0.22 | −0.1(−0.2 | 0.51 |
| | to 0.4) | | to 0.6) | | to 0.1) | | to 0.4) | | to 0.7) | | to 0.1) | |
| CGM HBGI | −2.7(−5.4 | 0.047 | −4.3(−7 | 0.002 | −1.1(−2.6 | 0.14 | −5.4(−8 | <0.001 | −1.6(−4.3 | 0.23 | −2.7(−4.7 | <0.001 |
| | to 6) | | to 1.6) | | to 0.4) | | to −2.7) | | to 1.1) | | to −1.2) | |
| Safety Metrics | | | | | | | | | | | | |
| Ketones | 0.07(0.03 | 0.007 | 0.05(0.01 | 0.014 | 0(−0.02 | 0.99 | 0.96(0.01 | 0.014 | 0.96(0.92 | 0.007 | 0(−0.03 | 0.61 |
| (mmol/L) | to 0.1) | | to 0.10) | | to 0.02) | | to 0.16) | | to 0.11) | | to 0.02) | |
| Technical | | | | | | | | | | | | |
| performance | | | | | | | | | | | | |
| metrics | | | | | | | | | | | | |
| Total Injected | −0.17 | 0.029 | −0.56 | 0.047 | 0.03 | 0.23 | −0.13 | 0.10 | −0.20 | 0.012 | 0.05(0 | 0.054 |
| Insulin | (−0.33 | | (−0.31 | | (−0.02 | | (−0.28 | | (−0.36 | | to 0.09) | |
| (IU/kg · day) | to −0.02) | | to 0) | | to 0.07) | | to 0.03) | | to −0.05) | | | |

*Plus-minus values are means ±SD. All the subjects were included in the model on an intention-to-treat basis. Missing data were handled by means of direct likelihood analyses. To convert the values for glucose to millimoles per liter, multiply by 0.05551. NA denotes not applicable. Baseline outcomes for 16 of the 32 subjects were not available.
The baseline glycated hemoglobin level was measured at the randomization visit.
§Differences were calculated as percentage points and were model-adjusted for the pre-randomization value of the HbA1c.
**Distributions were skewed and were thus modeled with the use of rank-based transformation. [symbol]
indicates data missing or illegible when filed

TABLE 5
Glycemic outcomes Daytime (7:00-23:00)

| | CIQ-EMPA | | | CIQ-NOEMPA | | | BIQ-EMPA | | | BIQ-NOEMPA | | |
|---|---|---|---|---|---|---|---|---|---|---|---|---|
| | n = 16 | | | n = 16 | | | n = 16 | | | n = 16 | | |
| Glycemic metrics | | | | | | | | | | | | |
| Mean CGM | 139 ± 19 | | | 154 ± 19 | | | 139 ± 26 | | | 163 ± 29 | | |
| glucose (mg/dL) | | | | | | | | | | | | |
| % CGM time <50 mg/dL | 0(0-0.1) | | | 0.3(0.1-0.4) | | | 0(0-0.4) | | | 0(0-0.5) | | |
| (<2.8 mmol/L)** | | | | | | | | | | | | |
| % CGM time <60 mg/dL | 0.4(0-0.5) | | | 0.7(0.3-1.6) | | | 0.5(0.1-0.9) | | | 0.4(0-1.9) | | |
| (<3.3 mmol/L)** | | | | | | | | | | | | |
| % CGM time <70 mg/dL | 1.2(0.6-1.7) | | | 1.6(0.7-3.8) | | | 1.7(0.9-2.7) | | | 1(0.6-5.2) | | |
| (<3.9 mmol/L)** | | | | | | | | | | | | |
| % CGM time | 56.7 ± 13.4 | | | 44.6 ± 11.6 | | | 58.1 ± 16.5 | | | 40.4 ± 15.1 | | |
| 70-140 mg/dL | | | | | | | | | | | | |
| (3.9-7.8 mmol/L) | | | | | | | | | | | | |
| % CGM time | 80.6 ± 10.5 | | | 70.8 ± 10 | | | 80 ± 14.5 | | | 63.5 ± 15.6 | | |
| 70-180 mg/dL | | | | | | | | | | | | |
| (3.9-10.0 mmol/L) | | | | | | | | | | | | |
| % CGM time >180 mg/dL | 15.8(6.2-26) | | | 24.6(18.5-36.4) | | | 11.3(5.3-22.4) | | | 26.5(18.6-47.7) | | |
| (>10.0 mmol/L)** | | | | | | | | | | | | |
| % CGM time >250 mg/dL | 1.2(0.4-3.7) | | | 3.8(1.8-7.4) | | | 1.7(0.1-4.4) | | | 6.6(2.7-13.9) | | |
| (>13.9 mmol/L)** | | | | | | | | | | | | |
| % CGM time >300 mg/dL | 0(0-0.6) | | | 1(0.1-2.4) | | | 0.1(0-0.8) | | | 1.5(0.2-5) | | |
| (>16.7 mmol/L)** | | | | | | | | | | | | |
| CGM SD (mg/dL) | 42.6 ± 9.7 | | | 52 ± 10 | | | 45 ± 14.6 | | | 58 ± 12 | | |
| CGM CV (%) | 30.4 ± 3.5 | | | 31.7 ± 4.7 | | | 35.7 ± 3.5 | | | 30.4 ± 3.5 | | |
| CGM LBGI** | 0.5(0.4-0.6) | | | 0.6(0.3-1.1) | | | 0.7(0.6-0.9) | | | 0.5(0.3-1.3) | | |
| CGM HBGI** | 3.4(1.9-5.5) | | | 5.2(4.1-7.9) | | | 2.8(1.6-4.9) | | | 6.2(4-10.7) | | |
| Safety Metrics | | | | | | | | | | | | |
| Ketones (mmol/L) | 0.20 ± 0.10 | | | 0.14 ± 0.11 | | | 0.20 ± 0.08 | | | 0.13 ± 0.06 | | |
| Technical | | | | | | | | | | | | |
| performance metrics | | | | | | | | | | | | |
| Injected Insulin | 0.41 ± 0.13 | | | 0.53 ± 0.19 | | | 0.37 ± 0.09 | | | 0.47 ± 0.17 | | |
| (IU/kg · day) | | | | | | | | | | | | |
| | CIQ-EMPA vs. | | BIQ-EMPA vs. | | CIQ-EMPA vs. | | CIQ-EMPA vs. | | BIQ-EMPA vs. | | CIQ-EMPA vs. | |
| | CIQ-NOEMPA | | BIQ-NOEMPA | | BIQ-NOEMPA | | BIQ-NOEMPA | | CIQ-NOEMPA | | BIQ-NOEMPA | |
| | Difference | p- | Difference | p- | Difference | p- | Difference | p- | Difference | p- | Difference | p- |
| | (95% CI)§ | value | (95% CI)§ | value | (95% CI)§ | value | (95% CI)§ | value | (95% CI)§ | value | (95% CI)§ | value |
| Glycemic metrics | | | | | | | | | | | | |
| Mean CGM | −14.6 | 0.086 | −23.8 | 0.007 | 0.2 | 0.95 | −23.6 | 0.007 | −14.9 | 0.08 | −8.9 | 0.029 |
| glucose (mg/dL) | (−31.5 | | (−40.6 | | (−7.7 | | (−40.4 | | (−31.6 | | (−16.9 | |
| | to 2.1) | | to −7) | | to 8.1) | | to −6.7) | | to −1.9 | | to −1) | |
| % CGM time <50 mg/dL | −0.2 | 0.19 | 0(−0.3 | 0.83 | 0.1(0.1 | 0.48 | 0(−0.3 | 0.78 | −0.3 | 0.075 | 0.2(0 | 0.033 |
| (<2.8 mmol/L) | (−0.5 | | to 0.2) | | to 0.3) | | to 0.3) | | (−0.6 | | to 0.5) | |
| | to 0.1) | | | | | | | | to 0) | | | |
| % CGM time <60 mg/dL | −0.7 | 0.16 | 0(−1 | 0.94 | −0.5 | 0.086 | −0.6 | 0.26 | −0.2 | 0.72 | 0.1(−0.5 | 0.63 |
| (<3.3 mmol/L) | (−1.7 to | | to 1) | | (−1.1 to | | (−1.6 to | | (−1.2 to | | to 0.7) | |
| | 0.3) | | | | 0.1) | | 0.4) | | 0.8) | | | |
| % CGM time <70 mg/dL | −1(−3.2 | 0.35 | 0.2(−1.9 | 0.81 | −1(−2 | 0.054 | −0.7 | 0.50 | 0(−2.2 | 0.98 | 0.3(−0.7 | 0.57 |
| (<3.9 mmol/L) | to 1.2) | | to 2.4) | | to 0) | | (−2.9 to | | to 2.1) | | to 1.3) | |
| | | | | | | | 1.4) | | | | | |
| % CGM time | 12(1.8 | 0.023 | 17.6 | 0.001 | −1.4 | 0.55 | 16.2 | 0.003 | 13(3.2 | 0.012 | 4.2 | 0.083 |
| 70-140 mg/dL | to 22.3) | | (7.4 to | | (−6.2 to | | (−6.2 to | | to 23.7) | | (−0.6 to | |
| (<3.9-7.8 mmol/L) | | | 27.9) | | 3.4) | | 3.4) | | | | 9) | |
| % CGM time | 9.9(0.6 | 0.037 | 16.5(7.3 | <0.001 | 0.6(−4.4 | 0.80 | 17.1(7.9 | <0.001 | 9.3(0 | 0.049 | 7.3(2.3 | 0.006 |
| <70-180 mg/dL | to 19.1) | | to 25.7) | | to 5.6) | | to 26.4) | | to 18.5) | | to 12.2) | |
| (3.9-10.0 mmol/L) | | | | | | | | | | | | |
| % CGM time | −10.3 | 0.045 | −16.7 | 0.002 | −0.8 | 0.72 | −17.6 | 0.001 | −9.5 | 0.064 | −7.3 | 0.003 |
| >180 mg/dL | (−20.3 to | | (−26.8 to | | (−5.4 to | | (−27.6 to | | (−19.5 to | | (−11.9 to | |
| (>10.0 mmol/L) | −0.2) | | −6.7) | | 3.8) | | −7.5) | | 0.6) | | −2.7) | |
| % CGM time | −3.8 | 0.12 | −7.6 | 0.003 | −1.6 | 0.24 | −9.2 | <0.001 | −2.2 | 0.37 | −5.4 | <0.001 |
| >250 mg/dL | (−8.7 to | | (−12.5 to | | (−4.5 to | | (−14.1 to | | (−7.1 to | | (−8.3 to | |
| (>13.9 mmol/L) | 11) | | −2.7) | | 1.2) | | −4.4) | | 2.7) | | −2.6 | |
| % CGM time | −1.3 | 0.20 | −2.7 | 0.015 | −0.9 | 0.21 | −3.6 | 0.001 | −0.4 | 0.69 | −2.2(−3.7 | 0.005 |
| >300 mg/dL | (−3.4 to | | (−4.8 to | | (−2.4 to | | (−5.7 to | | (−2.5 to | | to 0.7) | |
| (>16.7 mmol/L) | 0.8 | | −0.5) | | −0.5) | | −1.5) | | 1.7) | | | |
| CGM SD (mg/dL) | −9.3 | 0.030 | −13.2 | 0.003 | −2.3(−6.5 | 0.28 | −15.5 | <0.001 | −7.1 | 0.097 | −6.1 | 0.006 |
| | (−17.8 to | | (−21.6 to | | to 1.9) | | (−23.9 to | | (−15.5 to | | (−10.4 to | |
| | −0.9) | | 4.8) | | | | −7) | | 1.3) | | −1.9) | |
| CGM CV (%) | −3.3 | 0.022 | −4(−6.8 | 0.006 | −1.3(−3.1 | 0.13 | −5.3(−8.1 | <0.001 | −2(−4.8 | 0.16 | −2(−3.8 | 0.026 |
| | (−6.1 to | | to −1.2) | | to 0.4) | | to −2.5) | | to 0.8) | | to −0.3) | |
| | 0.5) | | | | | | | | | | | |
| CGM LBGI | 0(−0.5 | 0.97 | −0.3(−0.2 | 0.27 | −0.3(−0.5 | 0.011 | 0(−0.5 | 0.95 | 0.3(−0.2 | 0.23 | 0(−0.2 | 0.82 |
| | to 0.5 | | to 0.7) | | to 0.1) | | to 0.5) | | to 0.8) | | to 0.2) | |
| CGM HBGI | −2.2(−4.7 | 0.074 | −4(−6.5 | 0.002 | −6.3(−1.5 | 0.53 | −4.4(−6.8 | <0.001 | −1.9(−4.3 | 0.13 | −2.1(−3.3 | <0.001 |
| | to 0.2) | | to −1.6) | | to 0.8) | | to −1.9) | | to 0.6) | | to −1) | |
| Safety Metrics | | | | | | | | | | | | |
| Ketones (mmol/L) | 0.09(0.03 | 0.005 | 0.06(0 | 0.034 | 0.02 | 0.30 | 0.05(0.02 | 0.009 | 0.07(0.01 | 0.622 | 0(−0.04 | 0.75 |
| | to 0.15) | | to 0.12) | | (−0.62 to | | to 0.14) | | to 0.13) | | to 0.03) | |
| | | | | | 0.05) | | | | | | | |
| Technical | | | | | | | | | | | | |
| performance metrics | | | | | | | | | | | | |
| Total Injected | −0.11 | 0.057 | −0.09 | 0.092 | 0.04 | 0.047 | −0.06 | 0.32 | −0.15 | 0.047 | 0.05 | 0.010 |
| Insulin | (−0.22 to | | −0.21 to | | (0 to 0.08) | | (−0.17 to | | (−0.26 to | | (0.01 to | |
| (IU/kg · day) | 0) | | 0.02) | | | | 0.06) | | −0.04) | | 0.09) | |

*Plus-minus values are means ±SD. All the subjects were included in the model on an intention-to-treat basis. Missing data were handled by means of direct likelihood analyses. To convert the values for glucose to millimoles per liter, multiply by 0.05551. NA denotes not applicable.
Baseline outcomes for 16 of the 32 subjects were not available.
The baseline glycated hemoglobin level was measured at the randomization visit.
§Differences were calculated as percentage points and were model-adjusted for the pre-randomization value of the HbA1c.
**Distributions were skewed and were thus modeled with the use of rank-based transformation.

TABLE 6
Glycemic outcomes overnight.

| | CIQ-EMPA | | | CIQ-NOEMPA | | | BIQ-EMPA | | | BIQ-NOEMPA | | |
|---|---|---|---|---|---|---|---|---|---|---|---|---|
| | n = 16 | | | n = 16 | | | n = 16 | | | n = 16 | | |
| Glycemic metrics | | | | | | | | | | | | |
| Mean CGM | 133 ± 20 | | | 155 ± 20 | | | 145 ± 41 | | | 170 ± 41 | | |
| glucose (mg/dL) | | | | | | | | | | | | |
| % CGM time | 0(0-0.3) | | | 0.2(0-0.7) | | | 0(0-0.5) | | | 0.1(0-0.4) | | |
| <50 mg/dL | | | | | | | | | | | | |
| (<2.8 mmol/L)** | | | | | | | | | | | | |
| % CGM time | 0.3(0-0.5) | | | 0.8(0-1.7) | | | 0.5(0.1-1.1) | | | 0.3(0-1.7) | | |
| <60 mg/dL | | | | | | | | | | | | |
| (<3.3 mmol/L)** | | | | | | | | | | | | |
| % CGM time | 0.7(0-1.3) | | | 1.4(0.2-3.6) | | | 1.5(0.4-2.7) | | | 1.1(0-5.4) | | |
| <70 mg/dL | | | | | | | | | | | | |
| (<3.9 mmol/L)** | | | | | | | | | | | | |
| % CGM time | 64 ± 18 | | | 45 ± 15 | | | 54 ± 21 | | | 39 ± 20 | | |
| 70-140 mg/dL | | | | | | | | | | | | |
| (3.9-7.8 mmol/L) | | | | | | | | | | | | |
| % CGM time | 87 ± 13 | | | 72 ± 11 | | | 76 ± 21 | | | 59 ± 18 | | |
| 70-180 mg/dL | | | | | | | | | | | | |
| (3.9-10.0 mmol/L) | | | | | | | | | | | | |
| % CGM time | 6.3(3.3-11.1) | | | 27(19.1-32.2) | | | 16.2(7-30.8) | | | 41.6(17.2-48.5) | | |
| >180 mg/dL | | | | | | | | | | | | |
| (>10.0 mmol/L)** | | | | | | | | | | | | |
| % CGM time | 0(0-2) | | | 5(2.4-9.4) | | | 0.1(0-2.4) | | | 12.2(2.5-16.3) | | |
| >250 mg/dL | | | | | | | | | | | | |
| (>13.9 mmol/L)** | | | | | | | | | | | | |
| % CGM time | 0(0-0.4) | | | 0.8(0-4.4) | | | 0(0-0.2) | | | 3.3(0-10.5) | | |
| >300 mg/dL | | | | | | | | | | | | |
| (>16.7 mmol/L)** | | | | | | | | | | | | |
| CGM SD (mg/dL) | 34.2 ± 10.7 | | | 52.7 ± 12.3 | | | 44.5 ± 14 | | | 61.6 ± 14.7 | | |
| CGM CV (%) | 25.4 ± 5.4 | | | 34 ± 5 | | | 30.5 ± 4.2 | | | 36 ± 4.2 | | |
| CGM LBGI** | 0.4(0.2-0.5) | | | 0.5(0.2-1) | | | 0.7(0.4-1.4) | | | 0.3(0.1-1.8) | | |
| CGM HBGI** | 2.1(1.3-2.7) | | | 6.4(4.1-7) | | | 3.3(2.1-5.2) | | | 9.2(3.4-12) | | |
| Safety Metrics | | | | | | | | | | | | |
| Ketones (mmol/L)** | 0.17 ± 0.06 | | | 0.12 ± 0.00 | | | 0.19 ± 0.10 | | | 0.12 ± 0.00 | | |
| Technical | | | | | | | | | | | | |
| performance metrics | | | | | | | | | | | | |
| Injected Insulin | 0.11 ± 0.04 | | | 0.17 ± 0.09 | | | 0.09 ± 0.03 | | | 0.15 ± 0.08 | | |
| (IU/kg · day) | | | | | | | | | | | | |
| | CIQ-EMPA vs. | | BIQ-EMPA vs. | | CIQ-EMPA vs. | | CIQ-EMPA vs. | | BIQ-EMPA vs. | | CIQ-NOEMPA vs. | |
| | CIQ-NOEMPA | | BIQ-NOEMPA | | BIQ-EMPA | | BIQ-NOEMPA | | CIQ-NOEMPA | | BIQ-NOEMPA | |
| | Difference | p- | Difference | p- | Difference | p- | Difference | p- | Difference | p- | Difference | p- |
| | (95% CI)§ | value | (95% CI)§ | value | (95% CI)§ | value | (95% CI)§ | value | (95% CI)§ | value | (95% CI)§ | value |
| Glycemic metrics | | | | | | | | | | | | |
| Mean CGM | −21.5 | 0.066 | [symbol] | 0.037 | −11.7 | 0.004 | −36.2 | 0.001 | −9.8 | 0.39 | −14.7 | 0.027 |
| glucose (mg/dL) | (−44.5 to | | | | (−74.4 to | | (−59.2 to | | (−32.8 to | | (−27.6 to | |
| | 1.5) | | | | 1.2) | | −13.2) | | 13.2) | | −1.8) | |
| % CGM time | −0.1 | 0.39 | [symbol] | 0.071 | 0.2 | 0.002 | 0(−0.3 | 0.80 | −0.4 | 0.018 | 0.1 | 0.21 |
| <50 mg/dL | (−0.4 to | | | | (0.1 to | | to 0.3) | | (−0.7 to | | (0 to 0.2) | |
| (<2.8 mmol/L) | 0.7) | | | | 0.4) | | | | 0) | | | |
| % CGM time | −0.6 | 0.22 | 0.1(−0.1 | 0.75 | −0.4 | 0.072 | −0.3 | 0.52 | −0.1(−1 | 0.78 | 0.3 | 0.26 |
| <60 mg/dL | (−1.5 to | | to 1.1) | | (−9.5 to | | (−1.2 to | | to 0.8) | | (−0.2 to | |
| (<3.3 mmol/L) | 0.3) | | | | 0) | | 0.5) | | | | 0.8) | |
| % CGM time | −1.1 | 0.26 | 0.2(−1.7 | 0.81 | −1.5 | 0.013 | [symbol] | 0.23 | 0.4(−1.6 | 0.71 | −0.1(−1.3 | 0.82 |
| 70 mg/dL | (−3.1 to | | to 2.2) | | (−2.6 to | | | | to 2.4) | | to −1) | |
| (<3.9 mmol/L) | 0.9) | | | | −0.3) | | | | | | | |
| % CGM time | [symbol] | 0.007 | 15.4(2.1 | 0.024 | 10.2(3.6 | 0.004 | 25.5(12.3 | <0.001 | 8.6(−4.7 | 0.19 | 0.8(0.2 | 0.043 |
| 70-140 mg/dL | | | to 28.6) | | to 16.8) | | to 28.8) | | to 21.9) | | to 13.4) | |
| (3.9-7.8 mmol/L) | | | | | | | | | | | | |
| % CGM time | 15.5(3.8 | 0.011 | [symbol] | 0.006 | 10.1(4.8 | <0.001 | 27.7(26 | <0.001 | 4.8(−6.9 | 0.41 | 12.2(6.3 | <0.001 |
| 70-180 mg/dL | to 27.3) | | | | to 16.6) | | to 39.6) | | to 16.5) | | to 18) | |
| (3.9-10.0 mmol/L) | | | | | | | | | | | | |
| % CGM time | −14.6 | 0.021 | −18.2 | 0.005 | −9.5(−16 | 0.006 | −27.3 | <0.001 | −5.1 | 0.40 | −13 | <0.001 |
| >180 mg/dL | (−30 to | | (−20 to | | to −2.9) | | (−40 to | | (−17.5 to | | (−19.6 to | |
| (>10.0 mmol/L) | −2.3) | | −3.9) | | | | −13.3) | | 7.2) | | −6.3) | |
| % CGM time | −5.5 | 0.33 | −13.9 | <0.001 | −2.5 | 0.39 | −16.4 | <0.001 | −3(−10.3 | 0.41 | −10.9 | <0.001 |
| >250 mg/dL | (−12.8 to | | (−21.2 to | | (−8.3 to | | (−23.7 to | | to 4.2) | | (−16.7 to | |
| (>13.9 mmol/L) | 1.7) | | −6.6) | | 3.3) | | −9.1) | | | | −5.1) | |
| % CGM time | −2.5(−6.4 | 0.21 | −4.8 | [symbol] | −2.5 | 0.093 | −7.6 | <0.001 | −0.3 | 0.87 | −5.1 | 0.004 |
| >300 mg/dL | to 1.4) | | (−8.2 to | | (−6.1 to | | (−11.5 to | | (−3.6 to | | (−8.4 to | |
| (>16.7 mmol/L) | | | −0.9) | | 0.5) | | −3.7) | | 4.2) | | −1.8) | |
| CGM SD (mg/dL) | −18.3 | <0.001 | −17.2 | <0.00 | −10.2 | 0.001 | −27.1 | <0.001 | −8.2 | 0.003 | −9(−15 | 0.004 |
| | (−27.7 to | | (1.26 to | | (−16.1 to | | (36.7 to | | (−17.5 to | | to −3) | |
| | 9.1) | | 2.9) | | −4.2) | | −18) | | 1.1) | | | |
| CGM CV (%) | −8.3(−11.6 | <0.001 | −6(−9.3 | <0.001 | −5.1 | 0.002 | −11.1 | <0.001 | −3.1 | 0.064 | −3(−6 | 0.069 |
| | to −4.5) | | to −1.7) | | (−8.2 to | | (−14.3 to | | (−6.5 to | | to 0) | |
| | | | | | −2) | | −7.8) | | 0) | | | |
| CGM LBGI | 0.2(0.7 | 0.60 | 0.2(−0.3 | 0.51 | −0.5(−0.7 | 0.001 | −0.3(−0.8 | 0.26 | 0.3(−0.2 | 0.26 | −0.1(−0.4 | 0.35 |
| | to 0.3) | | to 0.7) | | to 0.2) | | to 0.2) | | to 0.8) | | to 0.1) | |
| CGM HBGI | −3.2(−6.8 | 0.073 | −6.2(−8.7 | 0.005 | −2.1(−4.4 | 0.064 | −7.3(−18.9 | <0.001 | −1.1(−4.6 | 0.54 | −4.1(−6.4 | <0.001 |
| | to 0.3) | | to −1.7) | | to 0.1) | | to −3.8) | | to −1.8) | | to −1.8) | |
| Safety Metrics | | | | | | | | | | | | |
| Ketones (mmol/L) | 0.06(0 | 0.020 | 0.04(0 | 0.12 | 0(−0.03 | 0.61 | 0.05(0 | 0.054 | 0.05(0 | 0.032 | 0(−0.05 | 0.66 |
| | to 0.0) | | to 0.10) | | to 0.04) | | to 0.10) | | to 0.10) | | to 0.03) | |
| Technical | | | | | | | | | | | | |
| performance metrics | | | | | | | | | | | | |
| Total Injected | [symbol] | 0.037 | −0.05 | 0.009 | 0.02(0 | 0.002 | 0(−0.009 | 0.077 | −0.00 | 0.002 | 0.82(0 | 0.020 |
| Insulin | | | (−0.11 to | | to 0.03) | | to 0.005) | | (−0.1 | | to 0.03) | |
| (IU/kg · day) | | | −0.02) | | | | | | to 0) | | | |

*Plus-minus values are means ±SD. All the subjects were included in the model on an intention-to-treat basis. Missing data were handled by means of direct likelihood analyses. To convert the values for glucose to millimoles per liter, multiply by 0.05551. NA denotes not applicable.
Baseline outcomes for 16 of the 32 subjects were not available.
The baseline glycated hemoglobin level was measured at the randomization visit.
§Differences were calculated as percentage points and were model-adjusted for the pre-randomization value of the HbA1c.
**Distributions were skewed and were thus modeled with the use of rank-based transformation. [symbol]
indicates data missing or illegible when filed

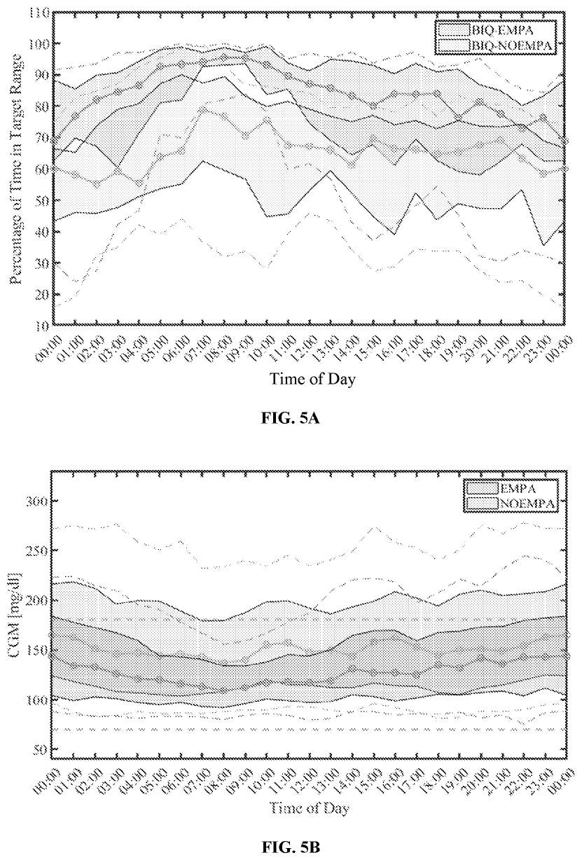

FIG. 4 presents daily profiles of CGM-based time in the target range 70-180 mg/dL TIR (FIG. 4A providing an envelope plot of percent TIR according to time of day) and CGM (FIG. 4B providing post-randomization hourly median sensor glucose with IQ envelope). Such figures illustrate the difference between AID with and without added SGLTi as SGLT2i. Overall, AID-EMPA showed an increase in TIR of +14.7 percentage points. Percentage times above 180 mg/dL and 250 mg/dL also weighed in favor of AID-EMPA with mean differences −12.3 and −4.9 percent points when compared to AID-NOEMPA, respectively. The infused insulin amounts demonstrated to be lower in AID-EMPA than in AID-NOEMPA by −0.17 IU/kg·day and −0.06 IU/kg·day during 24/7 and overnight periods, respectively, corresponding to an insulin reduction of 24% and 35.3%, respectively. For illustrative purposes as is shown with respect to FIG. 4B, “a” and “b” respectively denote upper and lower bounds of the target range (i.e., 70 and 180 mg/dL); encircled points ( [symbol] ) denote hourly median values; lower and upper bounds of EMPA and NOEMPA regions denote 25^{th} and 75^{th} percentiles; and corresponding regional dotted lines represent 10^{th} and 90^{th} percentiles. In these respects, such measurement indications can apply equally to FIGS. 4A and 5A-5B.

FIG. 5 mirrors FIG. 4 with respect to PLGS. A summary of glycemic outcomes is presented in Tables 4-6 above for both AID and PLGS with and without SGLTi as SGLT2i, for 24/7 (overall), daytime (7:00-23:00), and overnight periods.⁴² Mean±SD percent time in target range 70-180 mg/dL TIR during daytime (7:00 to 23:00 h) was 80±14.5 in the PLGS-EMPA versus 63.5±15.6 in the PLGS-NOEMPA (i.e., control) arm, translating into an increase of 16 percentage points in TIR for PLGS-EMPA versus PLGS-NOEMPA (see Table 3 and FIG. 5).

TABLE 3
Mirroring Primary and Secondary Outcomes for BIQ-EMPA vs BIQ-NOEMPA*

| | | | BIQ-EMPA vs. | |
|---|---|---|---|---|
| | BIQ- | BIQ- | BIQ-NOEMPA | |
| | EMPA | NOEMPA | Difference | p- |
| Outcome | n = 17 | n = 16 | (95% CI)§ | value |
| Primary: daytime glucose % Time in | 80 ± 14 | 63 ± 16 | 16.5(7.3 to 26) | <0.001 |
| range of 70 to 180 mg/dL (3.9-10.0 | | | | |
| mmol/L) | | | | |
| Secondary hierarchical outcomes | | | | |
| in prespecified order ¶ | | | | |
| 24/7 glucose level <70 mg/dL (<3.9 | 1.7(0.9-2.7) | 1.1(0.6-5.1) | 0.2(−1.7 to 2.1) | 0.82 |
| mmol/L) - median % time (IOR)** | | | | |
| 24/7 Mean glucose level - mg/dL | 141 ± 30 | 165 ± 32 | −24.1(−42.2 to −6) | NA |
| Daytime CGM SD - mg/dL | 44.9 ± 14.7 | 58.1 ± 12.1 | −13.2(−21.6 to −4.8) | NA |
| Daytime CGM CV - % | 30.4 ± 3.5 | 36 ± 3.5 | −4.0(−6.8 to −1.2) | NA |
| 24/7 LBGI - median (IQR)** | 0.7(0.6-1) | 0.5(0.3-1.3) | 0.1(−0.3 to 0.6) | NA |
| 24/7 HBGI - median (IQR)** | 3.3(1.8-4.7) | 7.6(3.8-10.6) | −3.7(−6.5 to −1) | NA |

*Plus-minus values are means ± SD. All the subjects were included in the model on an intention-to treat basis. Missing data were handled by means of direct likelihood analyses. To convert the values for glucose to millimoles per liter, multiply by 0.05551. NA denotes not applicable.
Baseline outcomes for 18 of the 35 subjects were not available.
The baseline glycated hemoglobin level was measured at the randomization visit.
§Differences were calculated as percentage points (the value in the treatment -EMPA- minus the value in the control -NOEMPA- group) and were model-adjusted for the pre-randomization value of the HbA1c.

As may be understood from the above, embodiments herein providing for the co-administration of selectively dosed formulation(s) of SGLTi and insulin therapy enhance opportunity for regulatory control of glycemia without promotion of, for instance, DKA.

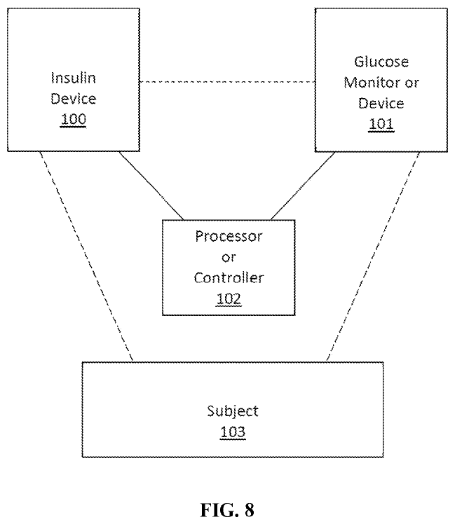

FIG. 8 is a high level functional block diagram of an embodiment of the present invention, or an aspect of an embodiment of the present invention. As shown in FIG. 8, a processor or controller 102 communicates with the glucose monitor or device 101, and optionally the insulin device 100. The glucose monitor or device 101 communicates with the subject 103 to monitor glucose levels of the subject 103. The processor or controller 102 may be configured to include all necessary hardware and/or software necessary to perform the required instructions to achieve relevant tasks (e.g., tasks associated with the ASM 620 and/or AAM 720), including required calculations. Optionally, the insulin device 100 communicates with the subject 103 to deliver insulin to the subject 103. The processor or controller 102 is configured to perform the required calculations. The glucose monitor 101 and the insulin device 100 may be implemented as a separate device or as a single device. The processor 102 can be implemented locally in the glucose monitor 101, the insulin device 100, or a standalone device (or in any combination of two or more of the glucose monitor, insulin device, or a stand along device). The processor 102 or a portion of the system can be located remotely such that the device is operated as a telemedicine device.

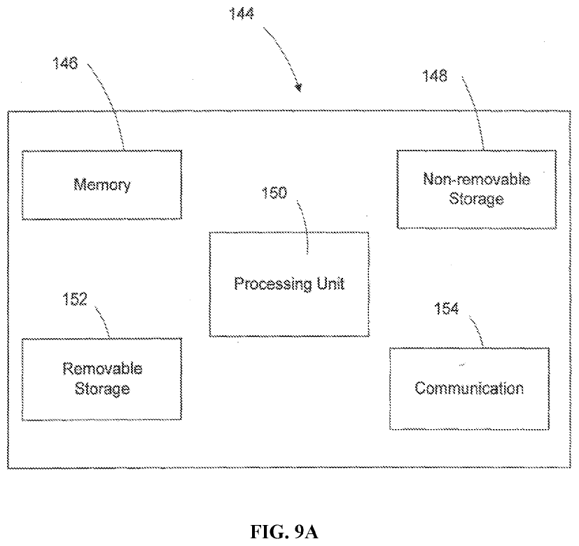

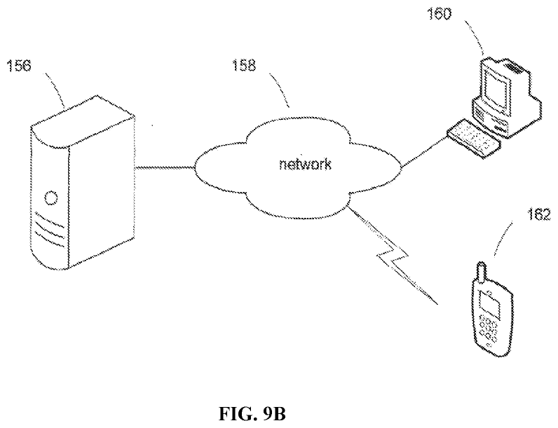

Referring to FIG. 9A, in its most basic configuration, computing device 144 typically includes at least one processing unit 150 and memory 146. Depending on the exact configuration and type of computing device, memory 146 can be volatile (such as RAM), non-volatile (such as ROM, flash memory, etc.) or some combination of the two.

Additionally, device 144 may also have other features and/or functionality. For example, the device could also include additional removable and/or non-removable storage including, but not limited to, magnetic or optical disks or tape, as well as writable electrical storage media. Such additional storage is the figure by removable storage 152 and non-removable storage 148. Computer storage media includes volatile and nonvolatile, removable and non-removable media implemented in any method or technology for storage of information such as computer readable instructions, data structures, program modules or other data. The memory, the removable storage and the non-removable storage are all examples of computer storage media. Computer storage media includes, but is not limited to, RAM, ROM, EEPROM, flash memory or other memory technology CDROM, digital versatile disks (DVD) or other optical storage, magnetic cassettes, magnetic tape, magnetic disk storage or other magnetic storage devices, or any other medium which can be used to store the desired information and which can accessed by the device. Any such computer storage media may be part of, or used in conjunction with, the device.

The device may also contain one or more communications connections 154 that allow the device to communicate with other devices (e.g. other computing devices). The communications connections carry information in a communication media. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode, execute, or process information in the signal. By way of example, and not limitation, communication medium includes wired media such as a wired network or direct-wired connection, and wireless media such as radio, RF, infrared and other wireless media. As discussed above, the term computer readable media as used herein includes both storage media and communication media.

In addition to a stand-alone computing machine, embodiments of the invention can also be implemented on a network system comprising a plurality of computing devices that are in communication with a networking means, such as a network with an infrastructure or an ad hoc network. The network connection can be wired connections or wireless connections. As a way of example, FIG. 9B illustrates a network system in which embodiments of the invention can be implemented. In this example, the network system comprises computer 156 (e.g. a network server), network connection means 158 (e.g. wired and/or wireless connections), computer terminal 160, and PDA (e.g. a smart-phone) 162 (or other handheld or portable device, such as a cell phone, laptop computer, tablet computer, GPS receiver, mp3 player, handheld video player, pocket projector, etc. or handheld devices (or non-portable devices) with combinations of such features). In an embodiment, it should be appreciated that the module listed as 156 may be glucose monitor device. In an embodiment, it should be appreciated that the module listed as 156 may be a glucose monitor device, artificial pancreas, and/or an insulin device (or other interventional or diagnostic device). Any of the components shown or discussed with FIG. 9B may be multiple in number. The embodiments of the invention can be implemented in anyone of the devices of the system. For example, execution of the instructions or other desired processing can be performed on the same computing device that is anyone of 156, 160, and 162. Alternatively, an embodiment of the invention can be performed on different computing devices of the network system. For example, certain desired or required processing or execution can be performed on one of the computing devices of the network (e.g. server 156 and/or glucose monitor device), whereas other processing and execution of the instruction can be performed at another computing device (e.g. terminal 160) of the network system, or vice versa. In fact, certain processing or execution can be performed at one computing device (e.g. server 156 and/or insulin device, artificial pancreas, or glucose monitor device (or other interventional or diagnostic device)); and the other processing or execution of the instructions can be performed at different computing devices that may or may not be networked. For example, the certain processing can be performed at terminal 160, while the other processing or instructions are passed to device 162 where the instructions are executed. This scenario may be of particular value especially when the PDA 162 device, for example, accesses to the network through computer terminal 160 (or an access point in an ad hoc network). For another example, software to be protected can be executed, encoded or processed with one or more embodiments of the invention. The processed, encoded or executed software can then be distributed to customers. The distribution can be in a form of storage media (e.g. disk) or electronic copy.

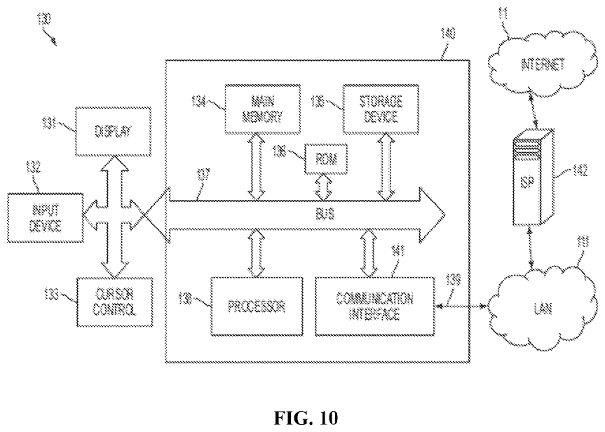

FIG. 10 is a block diagram that illustrates a system 130 including a computer system 140 and the associated Internet 11 connection upon which an embodiment may be implemented. Such configuration is typically used for computers (hosts) connected to the Internet 11 and executing a server or a client (or a combination) software. A source computer such as laptop, an ultimate destination computer and relay servers, for example, as well as any computer or processor described herein, may use the computer system configuration and the Internet connection shown in FIG. 10. The system 140 may be used as a portable electronic device such as a notebook/laptop computer, a media player (e.g., MP3 based or video player), a cellular phone, a Personal Digital Assistant (PDA), a glucose monitor device, an artificial pancreas, an insulin delivery device (or other interventional or diagnostic device), an image processing device (e.g., a digital camera or video recorder), and/or any other handheld computing devices, or a combination of any of these devices. Note that while FIG. 10 illustrates various components of a computer system, it is not intended to represent any particular architecture or manner of interconnecting the components; as such details are not germane to the present invention. It will also be appreciated that network computers, handheld computers, cell phones and other data processing systems which have fewer components or perhaps more components may also be used. The computer system of FIG. 10 may, for example, be an Apple Macintosh computer or Power Book, or an IBM compatible PC. Computer system 140 includes a bus 137, an interconnect, or other communication mechanism for communicating information, and a processor 138, commonly in the form of an integrated circuit, coupled with bus 137 for processing information and for executing the computer executable instructions. Computer system 140 also includes a main memory 134, such as a Random Access Memory (RAM) or other dynamic storage device, coupled to bus 137 for storing information and instructions to be executed by processor 138.

Main memory 134 also may be used for storing temporary variables or other intermediate information during execution of instructions to be executed by processor 138. Computer system 140 further includes a Read Only Memory (ROM) 136 (or other non-volatile memory) or other static storage device coupled to bus 137 for storing static information and instructions for processor 138. A storage device 135, such as a magnetic disk or optical disk, a hard disk drive for reading from and writing to a hard disk, a magnetic disk drive for reading from and writing to a magnetic disk, and/or an optical disk drive (such as DVD) for reading from and writing to a removable optical disk, is coupled to bus 137 for storing information and instructions. The hard disk drive, magnetic disk drive, and optical disk drive may be connected to the system bus by a hard disk drive interface, a magnetic disk drive interface, and an optical disk drive interface, respectively. The drives and their associated computer-readable media provide non-volatile storage of computer readable instructions, data structures, program modules and other data for the general purpose computing devices. Typically computer system 140 includes an Operating System (OS) stored in a non-volatile storage for managing the computer resources and provides the applications and programs with an access to the computer resources and interfaces. An operating system commonly processes system data and user input, and responds by allocating and managing tasks and internal system resources, such as controlling and allocating memory, prioritizing system requests, controlling input and output devices, facilitating networking and managing files. Non-limiting examples of operating systems are Microsoft Windows, Mac OS X, and Linux.

The term “processor” is meant to include any integrated circuit or other electronic device (or collection of devices) capable of performing an operation on at least one instruction including, without limitation, Reduced Instruction Set Core (RISC) processors, CISC microprocessors, Microcontroller Units (MCUs), CISC-based Central Processing Units (CPUs), and Digital Signal Processors (DSPs). The hardware of such devices may be integrated onto a single substrate (e.g., silicon “die”), or distributed among two or more substrates. Furthermore, various functional aspects of the processor may be implemented solely as software or firmware associated with the processor.

Computer system 140 may be coupled via bus 137 to a display 131, such as a Cathode Ray Tube (CRT), a Liquid Crystal Display (LCD), a flat screen monitor, a touch screen monitor or similar means for displaying text and graphical data to a user. The display may be connected via a video adapter for supporting the display. The display allows a user to view, enter, and/or edit information that is relevant to the operation of the system. An input device 132, including alphanumeric and other keys, is coupled to bus 137 for communicating information and command selections to processor 138. Another type of user input device is cursor control 133, such as a mouse, a trackball, or cursor direction keys for communicating direction information and command selections to processor 138 and for controlling cursor movement on display 131. This input device typically has two degrees of freedom in two axes, a first axis (e.g., x) and a second axis (e.g., y), that allows the device to specify positions in a plane.

The computer system 140 may be used for implementing the methods and techniques described herein. According to one embodiment, those methods and techniques are performed by computer system 140 in response to processor 138 executing one or more sequences of one or more instructions contained in main memory 134. Such instructions may be read into main memory 134 from another computer-readable medium, such as storage device 135. Execution of the sequences of instructions contained in main memory 134 causes processor 138 to perform the process steps described herein. In alternative embodiments, hard-wired circuitry may be used in place of or in combination with software instructions to implement the arrangement. Thus, embodiments of the invention are not limited to any specific combination of hardware circuitry and software.

The term “computer-readable medium” (or “machine-readable medium”) as used herein is an extensible term that refers to any medium or any memory, that participates in providing instructions to a processor, (such as processor 138) for execution, or any mechanism for storing or transmitting information in a form readable by a machine (e.g., a computer). Such a medium may store computer-executable instructions to be executed by a processing element and/or control logic, and data which is manipulated by a processing element and/or control logic, and may take many forms, including but not limited to, non-volatile medium, volatile medium, and transmission medium. Transmission media includes coaxial cables, copper wire and fiber optics, including the wires that comprise bus 137. Transmission media can also take the form of acoustic or light waves, such as those generated during radio-wave and infrared data communications, or other form of propagated signals (e.g., carrier waves, infrared signals, digital signals, etc.). Common forms of computer-readable media include, for example, a floppy disk, a flexible disk, hard disk, magnetic tape, or any other magnetic medium, a CD-ROM, any other optical medium, punch-cards, paper-tape, any other physical medium with patterns of holes, a RAM, a PROM, and EPROM, a FLASH-EPROM, any other memory chip or cartridge, a carrier wave as described hereinafter, or any other medium from which a computer can read.

Various forms of computer-readable media may be involved in carrying one or more sequences of one or more instructions to processor 138 for execution. For example, the instructions may initially be carried on a magnetic disk of a remote computer. The remote computer can load the instructions into its dynamic memory and send the instructions over a telephone line using a modem. A modem local to computer system 140 can receive the data on the telephone line and use an infra-red transmitter to convert the data to an infra-red signal. An infra-red detector can receive the data carried in the infra-red signal and appropriate circuitry can place the data on bus 137. Bus 137 carries the data to main memory 134, from which processor 138 retrieves and executes the instructions. The instructions received by main memory 134 may optionally be stored on storage device 135 either before or after execution by processor 138.

Computer system 140 also includes a communication interface 141 coupled to bus 137. Communication interface 141 provides a two-way data communication coupling to a network link 139 that is connected to a local network 111. For example, communication interface 141 may be an Integrated Services Digital Network (ISDN) card or a modem to provide a data communication connection to a corresponding type of telephone line. As another non-limiting example, communication interface 141 may be a local area network (LAN) card to provide a data communication connection to a compatible LAN. For example, Ethernet based connection based on IEEE802.3 standard may be used such as 10/100BaseT, 1000BaseT (gigabit Ethernet), 10 gigabit Ethernet (10 GE or 10 GbE or 10 GigE per IEEE Std 802.3ae-2002 as standard), 40 Gigabit Ethernet (40 GbE), or 100 Gigabit Ethernet (100 GbE as per Ethernet standard IEEE P802.3ba), as described in Cisco Systems, Inc. Publication number 1-587005-001-3 (6/99), “Internetworking Technologies Handbook”, Chapter 7: “Ethernet Technologies”, pages 7-1 to 7-38, which is incorporated in its entirety for all purposes as if fully set forth herein. In such a case, the communication interface 141 typically include a LAN transceiver or a modem, such as Standard Microsystems Corporation (SMSC) LAN91C111 10/100 Ethernet transceiver described in the Standard Microsystems Corporation (SMSC) data-sheet “LAN91C111 10/100 Non-PCI Ethernet Single Chip MAC+PHY” Data-Sheet, Rev. 15 (Feb. 20, 2004), which is incorporated in its entirety for all purposes as if fully set forth herein.

Wireless links may also be implemented. In any such implementation, communication interface 141 sends and receives electrical, electromagnetic or optical signals that carry digital data streams representing various types of information.

Network link 139 typically provides data communication through one or more networks to other data devices. For example, network link 139 may provide a connection through local network 111 to a host computer or to data equipment operated by an Internet Service Provider (ISP) 142. ISP 142 in turn provides data communication services through the world wide packet data communication network Internet 11. Local network 111 and Internet 11 both use electrical, electromagnetic or optical signals that carry digital data streams. The signals through the various networks and the signals on the network link 139 and through the communication interface 141, which carry the digital data to and from computer system 140, are exemplary forms of carrier waves transporting the information.

A received code may be executed by processor 138 as it is received, and/or stored in storage device 135, or other non-volatile storage for later execution. In this manner, computer system 140 may obtain application code in the form of a carrier wave. One or more aspects of the SGLTi-insulin co-administration may be implemented and utilized with the related processors, networks, computer systems, internet, and components and functions according to the schemes disclosed herein.

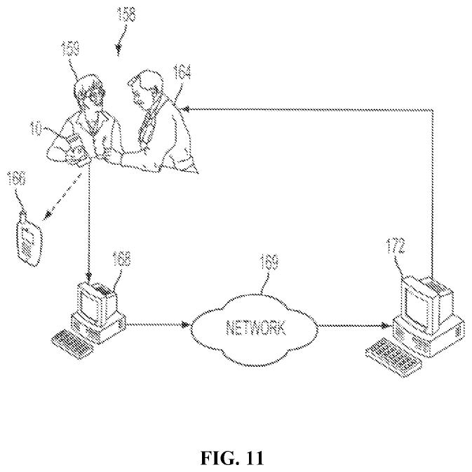

FIG. 11 illustrates a system in which one or more embodiments of the invention can be implemented using a network, or portions of a network or computers. Although the present invention glucose monitor, artificial pancreas or insulin device (or other interventional or diagnostic device) may be practiced without a network.

FIG. 11 diagrammatically illustrates an exemplary system in which examples of the invention can be implemented. In an embodiment the glucose monitor, artificial pancreas or insulin device (or other interventional or diagnostic device) may be implemented by the subject (or patient) locally at home or other desired location. However, in an alternative embodiment it may be implemented in a clinic setting or assistance setting. For instance, referring to FIG. 11, a clinic setup 158 provides a place for doctors (e.g. 164) or clinician/assistant to diagnose patients (e.g. 159) with diseases related with glucose and related diseases and conditions. A glucose monitoring device 10 can be used to monitor and/or test the glucose levels of the patient—as a standalone device. It should be appreciated that while only glucose monitor device 10 is shown in the figure, the system of the invention and any component thereof may be used in the manner depicted by FIG. 11. The system or component may be affixed to the patient or in communication with the patient as desired or required. For example the system or combination of components thereof—including a glucose monitor device 10 (or other related devices or systems such as a controller, and/or an artificial pancreas, an insulin pump (or other interventional or diagnostic device), or any other desired or required devices or components)—may be in contact, communication or affixed to the patient through tape or tubing (or other medical instruments or components) or may be in communication through wired or wireless connections. Such monitor and/or test can be short term (e.g. clinical visit) or long term (e.g. clinical stay or family). The glucose monitoring device outputs can be used by the doctor (clinician or assistant) for appropriate actions, such as insulin injection or food feeding for the patient, or other appropriate actions or modeling. Alternatively, the glucose monitoring device output can be delivered to computer terminal 168 for instant or future analyses. The delivery can be through cable or wireless or any other suitable medium. The glucose monitoring device output from the patient can also be delivered to a portable device, such as PDA 166. The glucose monitoring device outputs with improved accuracy can be delivered to a glucose monitoring center 172 for processing and/or analyzing. Such delivery can be accomplished in many ways, such as network connection 169, which can be wired or wireless.

In addition to the glucose monitoring device outputs, errors, parameters for accuracy improvements, and any accuracy related information can be delivered, such as to computer 168, and/or glucose monitoring center 172 for performing error analyses. This can provide a centralized accuracy monitoring, modeling and/or accuracy enhancement for glucose centers (or other interventional or diagnostic centers), due to the importance of the glucose sensors (or other interventional or diagnostic sensors or devices).

Examples of the invention can also be implemented in a standalone computing device associated with the target glucose monitoring device, artificial pancreas, and/or insulin device (or other interventional or diagnostic device). An exemplary computing device (or portions thereof) in which examples of the invention can be implemented is schematically illustrated in FIG. 9A.

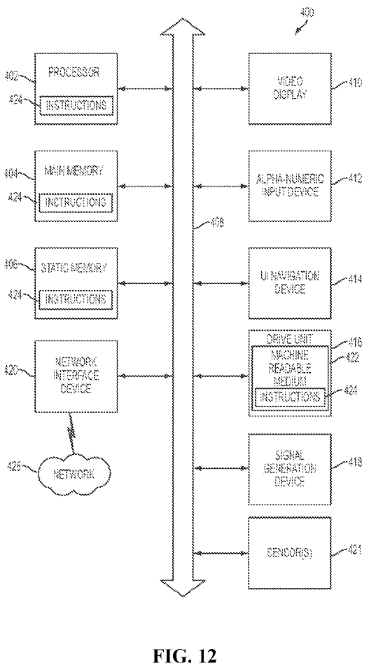

FIG. 12 is a block diagram illustrating an example of a machine upon which one or more aspects of embodiments of the present invention can be implemented.

Referring to FIG. 12, an aspect of an embodiment of the present invention includes, but is not limited thereto, a system, method, and computer readable medium that provides one or more aspects of the SGLTi-insulin co-administration discussed herein, such figure illustrating a block diagram of an example machine 400 upon which one or more embodiments (e.g., discussed methodologies) can be implemented (e.g., run).

Examples of machine 400 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service” (SaaS). For example, at least some of the operations can be performed by a group of computers (as examples of machines including processors), with these operations being accessible via a network (e.g., the Internet) and via one or more appropriate interfaces (e.g., Application Program Interfaces (APIs).)

Example embodiments (e.g., apparatus, systems, or methods) can be implemented in digital electronic circuitry, in computer hardware, in firmware, in software, or in any combination thereof. Example embodiments can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 400) and software architectures that can be deployed in example embodiments.

In an example, the machine 400 can operate as a standalone device or the machine 400 can be connected (e.g., networked) to other machines.

In a networked deployment, the machine 400 can operate in the capacity of either a server or a client machine in server-client network environments. In an example, machine 400 can act as a peer machine in peer-to-peer (or other distributed) network environments. The machine 400 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 400. Further, while only a single machine 400 is illustrated, the term “machine” shall also be taken to include any collection of machines that individually or jointly execute a set (or multiple sets) of instructions to perform any one or more of the methodologies discussed herein.

Example machine (e.g., computer system) 400 can include a processor 402 (e.g., a central processing unit (CPU), a graphics processing unit (GPU) or both), a main memory 404 and a static memory 406, some or all of which can communicate with each other via a bus 408. The machine 400 can further include a display unit 410, an alphanumeric input device 412 (e.g., a keyboard), and a user interface (UI) navigation device 411 (e.g., a mouse). In an example, the display unit 410, input device 412 and UI navigation device 414 can be a touch screen display. The machine 400 can additionally include a storage device (e.g., drive unit) 416, a signal generation device 418 (e.g., a speaker), a network interface device 420, and one or more sensors 421, such as a global positioning system (GPS) sensor, compass, accelerometer, or other sensor.

The storage device 416 can include a machine readable medium 422 on which is stored one or more sets of data structures or instructions 424 (e.g., software) embodying or utilized by any one or more of the methodologies or functions described herein. The instructions 424 can also reside, completely or at least partially, within the main memory 404, within static memory 406, or within the processor 402 during execution thereof by the machine 400. In an example, one or any combination of the processor 402, the main memory 404, the static memory 406, or the storage device 416 can constitute machine readable media.

While the machine readable medium 422 is illustrated as a single medium, the term “machine readable medium” can include a single medium or multiple media (e.g., a centralized or distributed database, and/or associated caches and servers) that configured to store the one or more instructions 424. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine readable media can include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 424 can further be transmitted or received over a communications network 426 using a transmission medium via the network interface device 420 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

### REFERENCES

The following patents, applications and publications as listed below and throughout this document are hereby incorporated by reference in their entirety herein, and which are not admitted to be prior art with respect to embodiments herein by inclusion in this section. Where applicable, citations herein refer to one or more of the documents below.
1. Kovatchev B. Automated closed-loop control of diabetes: the artificial pancreas. Bioelectron Med. 2018; 4(1):1-12.
2. Weisman A, Bai J-W, Cardinez M, Kramer C K, Perkins B A. Effect of artificial pancreas systems on glycaemic control in patients with type 1 diabetes: a systematic review and meta-analysis of outpatient randomised controlled trials. Lancet Diabetes Endocrinol. 2017; 5(7):501-512. doi:https://doi.org/10.1016/S2213-8587(17)30167-5
3. Bekiari E, Kitsios K, Thabit H, et al. Artificial pancreas treatment for outpatients with type 1 diabetes: Systematic review and meta-Analysis. BMJ. 2018; 361. doi:10.1136/bmj.kl310
4. Lal R A, Ekhlaspour L, Hood K, Buckingham B. Realizing a Closed-Loop (Artificial Pancreas) System for the Treatment of Type 1 Diabetes. Endocr Rev. 2019; 40(6):1521-1546. doi:10.1210/er.2018-00174
5. Breton M D, Kovatchev B P. One Year Real-World Use of the Control-IQ Advanced Hybrid Closed-Loop Technology. Diabetes Technol Ther. 2021; 23(9):601-608. doi:10.1089/dia.2021.0097
6. Brown S A, Kovatchev B P, Raghinaru D, et al. Six-Month Randomized, Multicenter Trial of Closed-Loop Control in Type 1 Diabetes. N Engl J Med. 2019; 381(18):1707-1717. doi:10.1056/nejmoal907863
7. Breton M D, Kanapka L G, Beck R W, et al. A Randomized Trial of Closed-Loop Control in Children with Type 1 Diabetes. N Engl J Med. 2020; 383(9):836-845. doi:10.1056/nejmoa2004736
8. Kovatchev B, Anderson S M, Raghinaru D, et al. Randomized Controlled Trial of Mobile Closed-Loop Control. Diabetes Care. 2020; 43:607-615.
9. Tauschmann M, Thabit H, Bally L, et al. Closed-loop insulin delivery in suboptimally controlled type 1 diabetes: a multicentre, 12-week randomised trial. Lancet. 2018; 392(10155):1321-1329.
10. Benhamou P Y, Franc S, Reznik Y, et al. Closed-loop insulin delivery in adults with type 1 diabetes in real-life conditions: a 12-week multicentre, open-label randomised controlled crossover trial. Lancet Digit Heal. 2019; 1(1):e17-e25. doi:10.1016/S2589-7500(19)30003-2
11. Collyns O J, Meier R A, Betts Z L, et al. Improved glycemic outcomes with medtronic minimed advanced hybrid closed-loop delivery: Results from a randomized crossover trial comparing automated insulin delivery with predictive low glucose suspend in people with type 1 diabetes. Diabetes Care. 2021; 44(4):969-975. doi:10.2337/dc20-2250
12. Bergenstal R M, Nimri R, Beck R W, et al. A comparison of two hybrid closed-loop systems in adolescents and young adults with type 1 diabetes (FLAIR): a multicentre, randomised, crossover trial. Lancet. 2021; 397(10270):208-219. doi:10.1016/S0140-6736(20)32514-9
13. Kovatchev B P, Kollar L, Anderson S M, et al. Evening and overnight closed-loop control versus 24/7 continuous closed-loop control for type 1 diabetes: a randomised crossover trial. Lancet Digit Heal. 2020; 2(2):e64-e73. doi:10.1016/S2589-7500(19)30218-3
14. Dove K, Piona C, Mutlu G Y, et al. Faster Compared with Standard Insulin Aspart during Day-And-Night Fully Closed-Loop Insulin Therapy in Type 1 Diabetes: A Double-Blind Randomized Crossover Trial. Diabetes Care. 2020; 43(1):29-36. doi:10.2337/dc19-0895
15. Boughton C K, Hartnell S, Thabit H, et al. Hybrid closed-loop glucose control with faster insulin aspart compared with standard insulin aspart in adults with type 1 diabetes: A double-blind, multicentre, multinational, randomized, crossover study. Diabetes, Obes Metab. 2021; 23(6):1389-1396. doi:10.1111/dom.14355
16. Hsu L, Buckingham B, Basina M, et al. Fast-Acting Insulin Aspart Use with the MiniMed 670G System. Diabetes Technol Ther. 2021; 23(1):1-7. doi:10.1089/dia.2020.0083
17. Garcia-Tirado J, Diaz J L, Esquivel-Zuniga R, et al. Advanced Closed-Loop Control System Improves Postprandial Glycemic Control Compared With a Hybrid Closed-Loop System Following Unannounced Meal. Diabetes Care. 2021; 44(10):2379-2387. doi:10.2337/dc21-0932
18. Sherr J L, Patel N S, Michaud C I, et al. Mitigating meal-related glycemic excursions in an insulin-sparing manner during closed-loop insulin delivery: The beneficial effects of adjunctive pramlintide and liraglutide. Diabetes Care. 2016; 39(7):1127-1134. doi:10.2337/dc16-0089
19. Renukuntla V S, Ramchandani N, Trast J, Cantwell M, Heptulla R A. Role of Glucagon-like peptide-1 analogue versus Amylin as an adjuvant therapy in type 1 diabetes in a closed loop setting with ePID algorithm. J Diabetes Sci Technol. 2014; 8(5):1011-1017. doi:10.1177/1932296814542153
20. Underland L J, Ilkowitz J T, Katikaneni R, Dowd A, Heptulla R A. Use of Sitagliptin with Closed-Loop Technology to Decrease Postprandial Blood Glucose in Type 1 Diabetes. J Diabetes Sci Technol. 2017; 11(3):602-610. doi:10.1177/1932296817699847
21. Haidar A, Tsoukas M A, Bernier-Twardy S, et al. A novel dual-hormone insulin- and-pramlintide artificial pancreas for type 1 diabetes: A randomized controlled crossover trial. Diabetes Care. 2020; 43(3):597-606. doi:10.2337/dc19-1922
22. Zinman B, Wanner C, Lachin J M, et al. Empagliflozin, cardiovascular outcomes, and mortality in type 2 diabetes. N Engl J Med. 2015; 373(22):2117-2128. doi:10.1056/nejmoal504720
23. Livingstone S J, Levin D, Looker H C, et al. Estimated life expectancy in a Scottish cohort with type 1 diabetes, 2008-2010. Jama. 2015; 313(1):37-44.
24. De Ferranti S D, De Boer I H, Fonseca V, et al. Type 1 diabetes mellitus and cardiovascular disease: a scientific statement from the American Heart Association and American Diabetes Association. Circulation. 2014; 130(13):1110-1130.
25. Morrish N J, Wang S-L, Stevens L K, Fuller J H, Keen H. Mortality and causes of death in the WHO Multinational Study of Vascular Disease in Diabetes. Diabetologia. 2001; 44(2):S14-S21.
26. Jorgensen M E, Almdal T P, Carstensen B. Time trends in mortality rates in type 1 diabetes from 2002 to 2011. Diabetologia. 2013; 56(11):2401-2404.
27. Secrest A M, Becker D J, Kelsey S F, LaPorte R E, Orchard T J. All-cause mortality trends in a large population-based cohort with long-standing childhood-onset type 1 diabetes: the Allegheny County type 1 diabetes registry. Diabetes Care. 2010; 33(12):2573-2579.
28. Kanter J E, Shao B, Kramer F, et al. Increased apolipoprotein C3 drives cardiovascular risk in type 1 diabetes. J Clin Invest. 2019; 129(10):4165-4179.
29. Saisho Y. Glycemic variability and oxidative stress: a link between diabetes and cardiovascular disease?Int J Mol Sci. 2014; 15(10):18381-18406.
30. Finne P, Reunanen A, Stenman S, Groop P-H, Granhagen-Riska C. Incidence of end-stage renal disease in patients with type 1 diabetes. Jama. 2005; 294(14):1782-1787.
31. McGuire D K, Shih W J, Cosentino F, et al. Association of SGLT2 inhibitors with cardiovascular and kidney outcomes in patients with type 2 diabetes: a meta-analysis. JAMA Cardiol. 2021; 6(2):148-158.
32. Rosenstock J, Marquard J, Laffel L M, et al. Empagliflozin as adjunctive to insulin therapyin type 1 diabetes: The EASE trials. Diabetes Care. 2018; 41(12):2560-2569. doi:10.2337/dc18-1749
33. van Raalte D H, Bjornstad P, Persson F, et al. The impact of sotagliflozin on renal function, albuminuria, blood pressure, and hematocrit in adults with type 1 diabetes. Diabetes Care. 2019; 42(10):1921-1929.
34. Baker C, Wason S, Banks P, et al. Dose-dependent glycometabolic effects of sotagliflozin on type 1 diabetes over 12 weeks: The inTandem4 trial. Diabetes, Obes Metab. 2019; 21(11):2440-2449.
35. Rodbard H W, Peters A L, Slee A, Cao A, Traina S B, Alba M. The effect of canagliflozin, a sodium glucose cotransporter 2 inhibitor, on glycemic end points assessed by continuous glucose monitoring and patient-reported outcomes among people with type 1 diabetes. Diabetes Care. 2017; 40(2):171-180.
36. Henry R R, Thakkar P, Tong C, Polidori D, Alba M. Efficacy and safety of canagliflozin, a sodium-glucose cotransporter 2 inhibitor, as add-on to insulin in patients with type 1 diabetes. Diabetes Care. 2015; 38(12):2258-2265. doi:10.2337/dcl5-1730
37. Siebel S, Galderisi A, Patel N S, Carria L R, Tamborlane W V, Sherr J L. Reversal of ketosis in type 1 diabetes is not adversely affected by SGLT2 inhibitor therapy. Diabetes Technol\& Ther. 2019; 21(3):101-104.
38. Patel N S, Van Name M A, Cengiz E, et al. Altered patterns of early metabolic decompensation in type 1 diabetes during treatment with a SGLT2 inhibitor: an insulin pump suspension study. Diabetes Technol\& Ther. 2017; 19(11):618-622.
39. Biester T, Muller I, von dem Berge T, et al. Add-on therapy with dapagliflozin under full closed loop control improves time in range in adolescents and young adults with type 1 diabetes: The DAPADream study. Diabetes, Obes Metab. 2020; 23(2):599-608. doi:10.1111/dom.14258
40. Haidar A, Yale J F, Lovblom L E, et al. Reducing the need for carbohydrate counting in type 1 diabetes using closed-loop automated insulin delivery (artificial pancreas) and empagliflozin: A randomized, controlled, non-inferiority, crossover pilot trial. Diabetes, Obes Metab. 2021; 23(6):1272-1281. doi:10.1111/dom.14335
41. Kovatchev B P. Metrics for glycaemic control—from HbA 1c to continuous glucose monitoring. Nat Rev Endocrinol. 2017; 13(7):425-436.
42. Maahs D M, Buckingham B A, Castle J R, et al. Outcome measures for artificial pancreas clinical trials: A consensus report. Diabetes Care. 2016; 39(7):1175-1179. doi:10.2337/dc15-2716

Although the present embodiments have been described in detail, those skilled in the art will understand that various changes, substitutions, variations, enhancements, nuances, gradations, lesser forms, alterations, revisions, improvements and knock-offs of the embodiments disclosed herein may be made without departing from the spirit and scope of the embodiments in their broadest form.

What is claimed is:

1. A processor-implemented method for controlling a glucose level in a subject having Type 1 diabetes, the method comprising:
providing the subject with an initial dose of sodium-glucose cotransporter inhibitor (SGLTi) lower than a dosing standardized, solely, for treatment of hyperglycemia;
analyzing the glucose level, in real-time, via an automated insulin delivery system comprising continuous glucose monitoring (CGM);
providing insulin to the subject via the automated insulin delivery system; and
adjusting the insulin delivery, by the automated insulin delivery system, to the subject to maintain the glucose level within a target glucose time-in-range (TIR).

2. The method according to claim 1, wherein:
the SGLTi comprises at least one of (a) a sodium-glucose cotransporter type 2 (SGLT2) inhibitor and (b) a combination sodium-glucose cotransporter type 1 (SGLT1) and SGLT2 inhibitor.

3. The method according to claim 2, wherein:
the initial dose of SGLTi is about 25% to about 50% of the dosing standardized, solely, for treatment of hyperglycemia.

4. The method according to claim 1, wherein:
the providing the subject with an initial dose of SGLTi comprises providing a daily dose of five (5) mg of SGLT2i empagliflozin (EMPA).

5. The method according to claim 1, further comprising:
adjusting the initial dose of SGLTi and/or the insulin delivery in response to detecting, from the CGM data, a postprandial excursion.

6. The method according to claim 5, wherein:
a magnitude of the postprandial excursion is measured according to an Area under the Curve (AUC), and measurement for any adjustment to the initial dose of SGLTi is given by:
$${SGLTi}_{dose} = \{ \begin{matrix}initial dose & if μ ( AUC ) \leq {AUC}_{th} \\ scale - up & if AUC > {AUC}_{th} \\ scale - down & if K \geq {K}_{th} or {N}_{h} > 2\end{matrix} ,$$
where μ(AUC) represents the averaged area-under-the curve over the last Nₜᵣ days according to CGM trace, AUCₜₕ represents a predetermined design AUC threshold,
$$K = g ( IOB , {\{ CGM \}}_{k - T}^{k} , {\{ {u}_{i} \}}_{k - T}^{k} )$$
represents a daily ketone estimation with Kₜₕ=1.5 mmol/L, and Nₕ is the number of hypoglycemic episodes over the Nₜᵣ days corresponding to the time spent below 70 mg/dL.

7. A system for controlling a glucose level in a subject having Type 1 diabetes, comprising:
a processor; and
a processor-readable memory including processor-executable instructions for, in response to providing the subject with an initial dose of sodium-glucose cotransporter inhibitor (SGLTi) lower than a dosing standardized, solely, for treatment of hyperglycemia,
analyzing the glucose level, in real-time, via an automated insulin delivery system comprising continuous glucose monitoring (CGM);
providing insulin to the subject via the automated insulin delivery system; and
adjusting the insulin delivery, by the automated insulin delivery system, to the subject to maintain the glucose level within a target glucose time-in-range (TIR).

8. The system according to claim 7, wherein:
the SGLTi comprises at least one of (a) a sodium-glucose cotransporter type 2 (SGLT2) inhibitor and (b) a combination sodium-glucose cotransporter type 1 (SGLT1) and SGLT2 inhibitor.

9. The system according to claim 8, wherein:
the initial dose of SGLTi is about 25% to about 50% of the dosing standardized, solely, for treatment of hyperglycemia.

10. The system according to claim 7, wherein:
the initial dose of SGLTi comprises a daily dose of five (5) mg of SGLT2 empagliflozin (EMPA).

11. The system according to claim 7, further comprising:
the initial dose of SGLTi and/or the insulin delivery are adjusted in response to detecting, from the CGM data, a postprandial excursion.

12. The system according to claim 11, wherein:
a magnitude of the postprandial excursion is measured according to an Area under the Curve (AUC), and measurement for any adjustment to the initial dose of SGLTi is given by:
$${SGLTi}_{dose} = \{ \begin{matrix}initial dose & if μ ( AUC ) \leq {AUC}_{th} \\ scale - up & if AUC > {AUC}_{th} \\ scale - down & if K \geq {K}_{th} or {N}_{h} > 2\end{matrix} ,$$
where μ(AUC) represents the averaged area-under-the curve over the last Nₜᵣ days according to CGM trace, AUCₜₕ represents a predetermined design AUC threshold,
$$K = g ( IOB , {\{ CGM \}}_{k - T}^{k} , {\{ {u}_{i} \}}_{k - T}^{k} )$$
represents a daily ketone estimation with Kₜₕ=1.5 mmol/L, and Nₕ is the number of hypoglycemic episodes over the Nₜᵣ days corresponding to the time spent below 70 mg/dL.

13. A non-transient computer-readable medium having stored thereon computer-executable instructions for controlling a glucose level in a subject having Type 1 diabetes, said instructions comprising instructions causing a computer to, in response to providing the subject with an initial dose of sodium-glucose cotransporter inhibitor (SGLTi) lower than a dosing standardized, solely, for treatment of hyperglycemia:
analyze the glucose level, in real-time, via an automated insulin delivery system comprising continuous glucose monitoring (CGM);
provide insulin to the subject via the automated insulin delivery system; and
adjust the insulin delivery, by the automated insulin delivery system, to the subject to maintain the glucose level within a target glucose time-in-range (TIR).

14. The medium according to claim 13, wherein:
the SGLTi comprises at least one of (a) a sodium-glucose cotransporter type 2 (SGLT2) inhibitor and (b) a combination sodium-glucose cotransporter type 1 (SGLT1) and SGLT2 inhibitor.

15. The medium according to claim 14, wherein:
the initial dose of SGLTi is about 25% to about 50% of the dosing standardized, solely, for treatment of hyperglycemia.

16. The medium according to claim 13, wherein:
the initial dose of SGLTi comprises a daily dose of five (5) mg of SGLT2 empagliflozin (EMPA).

17. The medium according to claim 13, wherein:
the instructions further comprise adjusting the initial dose of SGLTi and/or the insulin delivery in response to detecting, from the CGM data, a postprandial excursion.

18. The medium according to claim 17, wherein:
a magnitude of the postprandial excursion is measured according to an Area under the Curve (AUC), and measurement for any adjustment to the initial dose of SGLTi is given by:
$${SGLTi}_{dose} = \{ \begin{matrix}initial dose & if μ ( AUC ) \leq {AUC}_{th} \\ scale - up & if AUC > {AUC}_{th} \\ scale - down & if K \geq {K}_{th} or {N}_{h} > 2\end{matrix} ,$$
where μ(AUC) represents the averaged area-under-the curve over the last Nₜᵣ days according to CGM trace, AUCₜₕ represents a predetermined design AUC threshold,
$$K = g ( IOB , {\{ CGM \}}_{k - T}^{k} , {\{ {u}_{i} \}}_{k - T}^{k} )$$
represents a daily ketone estimation with Kₜₕ=1.5 mmol/L, and
Nₕ is the number of hypoglycemic episodes over the Nₜᵣ days corresponding to the time spent below 70 mg/dL.
